(19) United States
(12) Patent Application Publication
(10) Pub. No.: US 2025/0188432 A1
(43) Pub. Date: Jun. 12, 2025

(54) REVERSAL OF EXHAUSTION OF HIV-1 SPECIFIC CTLS BY CRISPR-MEDIATED DISRUPTION OF PD-1 GENE

(71) Applicant: Temple University-Of The Commonwealth System of Higher Education, Philadelphia, PA (US)

(72) Inventor: RAFAL KAMINSKI, Philadelphia, PA (US)

(21) Appl. No.: 18/978,609

(22) Filed: Dec. 12, 2024

Related U.S. Application Data
(60) Provisional application No. 63/608,926, filed on Dec. 12, 2023.

(51) Int. Cl. C12N 9/22; A61K 48/00; C12N 15/11; C12N 15/86; C12N 15/90

(52) U.S. Cl. CPC C12N 9/22; C12N 15/11; C12N 15/86; C12N 15/907; A61K 48/00; C12N 2310/20; C12N 2750/14143

(57) ABSTRACT

Described herein are systems and methods that demonstrate that the inhibition of PD-1 by Cas9-based gene editing in cytotoxic T lymphocytes reduces CTL exhaustion in chronic HIV-1 infection.

### CROSS-REFERENCE TO RELATED APPLICATIONS

This application claims the benefit of priority to U.S. Provisional Application No. 63/608,926, filed Dec. 12, 2023, the contents of which is hereby incorporated by reference in its entirety.

### STATEMENT REGARDING FEDERALLY SPONSORED RESEARCH OR DEVELOPMENT

This invention was made with government support under UM1 AI164568 awarded by the National Institutes of Health. The government has certain rights in the invention.

### REFERENCE TO A SEQUENCE LISTING SUBMITTED AS AN XML FILE

This Sequence Listing written in the XML file titled “206017-0261-00US_SequenceListing.xml” in XML format, with a creation date of Dec. 11, 2024, and 27,726 bytes in size, is hereby incorporated by reference in its entirety.

### BACKGROUND OF THE INVENTION

CTLs (cytotoxic T lymphocytes) mediate strong suppression of HIV infection. Unfortunately, chronic HIV-1 infection drives functional exhaustion of viral antigen-specific CTLs with elevated and sustained expression of the immune checkpoint receptors such as PD-1. This functional impairment, including lack of antigen-driven proliferation and poor cytolytic capacity, cannot be fully restored by ART (antiretroviral therapy).

Thus, there is a need in the art for improved compositions and methods for preventing the exhaustion of viral antigen-specific CTLs. This invention satisfies this unmet need.

### SUMMARY OF THE INVENTION

In some embodiments the present invention provides a genome editing system comprising: a) a Cas peptide or a nucleic acid encoding said Cas peptide; and b) at least one guide RNA (gRNA) molecule comprising a targeting domain that is complementary to a target sequence of a PD-1 gene or at least one nucleic acid encoding said at least one gRNA molecule. In some embodiments, the Cas peptide is a Cas9 peptide. In some embodiments, the Cas9 peptide is a Staphylococcus aureus Cas9 (SaCas9) peptide. In some embodiments, the SaCas9 peptide is an adeno-associated virus (AAV)-compatible SaCas9.

In some embodiments, said at least one gRNA molecule comprises a nucleotide sequence having at least 95% identity to at least one nucleotide sequence selected from the group consisting of SEQ ID NOs:1-5. In some embodiments, said at least one gRNA molecule comprises at least one nucleotide sequence selected from the group consisting of SEQ ID NOs:1-5.

In some embodiments, said at least one gRNA molecule comprises at least two gRNA molecules, wherein said at least two gRNA molecules comprise at least two sequences selected from the group consisting of SEQ ID NOs:1-5. In some embodiments, said at least two gRNA molecules comprise at least a first gRNA molecule comprising SEQ ID NO:1 and a second gRNA molecule comprising SEQ ID NO:3.

In some embodiments, the present invention provides a plasmid encoding a genome editing system of the present invention, comprising a nucleotide sequence encoding said Cas peptide and at least one nucleotide sequence encoding said at least one gRNA molecule. In some embodiments, said Cas peptide is an AAV-compatible SaCas9 peptide.

In some embodiments, said at least one gRNA molecule comprises a nucleotide sequence having at least 95% identity to at least one nucleotide sequence selected from the group consisting of SEQ ID NOs:1-5. In some embodiments, said at least one gRNA molecule comprises a nucleotide sequence selected from the group consisting of SEQ ID NOs:1-5.

In some embodiments, the plasmid encodes at least two gRNA molecules, wherein said at least two gRNA molecules comprise at least two sequences selected from the group consisting of SEQ ID NOs:1-5. In some embodiments, said at least two gRNA molecules comprise at least a first gRNA molecule comprising SEQ ID NO:1 and a second gRNA molecule comprising SEQ ID NO:3.

In some embodiments, the present invention provides an adeno-associated virus (AAV) comprising a plasmid of the present invention. In some embodiments, the AAV is an AAV6 or an AAV9 serotype.

In some embodiments, the present invention provides a method of decreasing the expression of a PD-1 gene in a cell, comprising contacting the cell with the genome editing system of claim 1.

In some embodiments, the present invention provides a method of reducing cytotoxic T lymphocyte (CTL) exhaustion in a subject comprising administering to the subject an AAV of the present invention.

In some embodiments, the present invention provides a method of treating or preventing a disease or disorder in a subject, comprising administering to the subject an AAV of the present invention. In some embodiments, the subject is infected with a pathogen selected from the group consisting of: human immunodeficiency virus 1 (HIV-1), hepatitis B virus (HBV), hepatitis C virus (HCV), human T-lymphotropic virus 1 (HTLV-1), influenza, Mycobacterium tuberculosis, a Plasmodium species, a Listeria species, a Toxoplasma species, and a Leishmania species.

### BRIEF DESCRIPTION OF THE DRAWINGS

The following detailed description of embodiments of the invention will be better understood when read in conjunction with the appended drawings. It should be understood that the invention is not limited to the precise arrangements and instrumentalities of the embodiments shown in the drawings.

FIG. 1 depicts targeting of PD-1 in HIV-1 specific CTLs.

FIG. 2 depicts a Schematic of the chromosomal location of the human PDCD1 gene encoding PD-1, its structure, and the position of selected CRISPR gRNA target sites. TSS—transcription start site, TTS—transcription termination site, ATG—codon START, TGA—codon STOP, coding sequence is highlighted in yellow, gRNA protospacers in green and PAM sequences in red.

FIG. 3, comprising FIG. 3A through FIG. 3E, depicts results from example experiments demonstrating the construction of guide RNAs. FIG. 3A depicts agarose gel analysis of PCR amplification of the exon 1-exon 3 region of the PDCD1 gene. Blue arrow points to full-length amplicon. FIG. 3B depicts sanger sequencing verification of the truncated amplicons representing CRISPR-cleaved/end-joined products of the PDCD1 gene editing. gRNAs target sites are highlighted in green (g1) and yellow (g3), PAM in red. FIG. 3C depicts a table showing % of the PDCD1 gene excision calculated from ratio (truncated) to (truncated+full-length) using data from two independent experiments (ImageJ). FIG. 3D depicts representative comparison of off-sight incorporation by SaCas9 with different guide RNAs. FIG. 3E depicts representative GuideSeq results with g1 and g3.

FIG. 4 depicts results from flow cytometry analysis of PD-1 staining. Most guide RNAs resulted in decreased PD-1 expression.

FIG. 5, comprising FIG. 5A through FIG. 5E, depicts the results of example gene editing experiments. FIG. 5A depicts a schematic of the experimental layout. FIG. 5B depicts agarose gel analysis of PCR amplification of the exon 1-exon 3 region of the PDCD1 gene. Blue arrow points to full-length (upper) and truncated (lower) amplicons. FIG. 5C depicts flow-cytometry histograms showing surface expression of PD-1 in unstimulated and CD3/CD28 induced CD8 T cells from Donor3. FIG. 5D depicts a bar plot summarizing % of cells expressing PD-1 for all three donors. FIG. 5E depicts Sanger sequencing verification of the truncated amplicons representing CRISPR-cleaved/end-joined products of the PDCD1 gene editing.

FIG. 6, comprising FIG. 6A through FIG. 6C, depicts the CRISPR-PD-1-g1+g3 AAV delivery vector and vaccination experiments. FIG. 6A depicts a map of the CRISPR-PD-1-g1+g3 AAV delivery vector. FIG. 6B depicts agarose gel analysis of RT-PCR verifying expression of SaCas9 mRNA and gRNAs PD-1-g1 and PD-1-g3. FIG. 6C depicts an agarose gel of PCR amplification of the exon1-exon3 region of the PDCD1 gene in control (pX601-no gRNA) and CRISPR-PD-1-g1+g3 treated (pX601-PD-1-g1+g3) K562 myeloid cell line cells.

FIG. 7, comprising FIG. 7A through FIG. 7F, depicts the results of example experiments to develop in vitro assay using primary immune cells from HIV+ donors. FIG. 7A depicts a schematic of the experimental layout. FIG. 7B depicts light microscopy images of CD8⁺ T cells, MDMs, and co-cultures. FIG. 7C depicts representative quantification of PD1-FITC-positive cells from FIG. 7B. FIG. 7D depicts agarose gel analysis and Sanger sequencing analysis of PCR amplification of the exon 1-exon 3 region of the PDCD1 gene. Blue arrows to full-length (upper) and truncated(lower)amplicons. Sanger sequencing results depict representative truncation of the amplicons representing CRISPR-cleaved/end-joined products of the PDCD 1 gene editing. gRNA start sites are highlighted in green (g1) and yellow (g3), PAM in red. FIG. 7E depicts immunolabeling/flow-cytometry analysis of PD-1 expression in CD8⁺ T cells after 1 week of co-culture (left). Summary data from three donors are shown on the right. FIG. 7F depicts representative flow cytometry FarRed/PI staining analysis for cytotoxic killing assay (left). Summary data from three donors are shown on the right.

FIG. 8, comprising FIGS. 8A and 8B. depicts results from an exemplary CTL-mediated killing assay of PD-1 CRISPR editing. FIG. 8A depicts representative agarose gel analysis and Sanger sequencing verification of PCR amplification of the exon 1-exon 3 region of the PDCD1 gene. Blue arrow points to full-length (upper) and truncated (lower)amplicons. The Sanger sequencing verification demonstrates the truncated amplicons representing CRISPR-cleaved/end-joined products of the PDCD 1 gene editing. FIG. 8B depicts representative flow cytometry results demonstrating the population of cells expressing Gag p24.

FIG. 9 depicts results from an exemplary off-target analysis of PD-1 guides g1 and g3.

FIG. 10, comprising FIG. 10A through FIG. 10F, depicts representative results of PD-1 knockdown in CD8⁺ T cells from healthy donors and their cytotoxic function against infected CD4⁺ T cells. FIG. 10A depicts a schematic representation of an experimental setup. FIG. 10B depicts a representative image of agarose gel analysis of PCR amplification of the exon1-exon3 region of the PDCD1 gene. FIG. 10C depicts representative quantification of PD-1 expression in CD8⁺ T cells by immunolabeling and flow cytometry before a cytotoxic killing assay. FIG. 10D depicts representative quantification of PD-1 dead CD4⁺ T cells by flow cytometry after a cytotoxic killing assay. FIG. 10E depicts representative quantification of GFP-positive cells by flow cytometry after a cytotoxic killing assay. FIG. 10F depicts representative flow cytometry results from a cytotoxic killing assay.

FIG. 11, comprising FIG. 11A through FIG. 11F, depicts representative in vivo results demonstrating AAV-delivery of SaCas9/PD-1 g1+g3 gRNAs in MISTRG-6-15 mice. FIG. 11A depicts a schematic representation of an experimental setup. FIG. 11B depicts representative quantification of PD1 expression in CD3-positive T lymphocytes isolated from spleens of treated animals. FIG. 11C depicts representative quantification of PD1 expression in CD3-positive T lymphocytes isolated from livers of treated animals. FIG. 11D depicts representative quantification of SaCas9 transcription in cells isolated from spleens and livers of treated animals. FIG. 11E depicts the quantification of SaCas9 expression in cells isolated from spleens and livers of treated animals. FIG. 11F depicts the verification of PD-1 truncation by agarose gel and Sanger sequencing.

FIG. 12, comprising FIG. 12A and FIG. 12B, depicts representative in vivo delivery of SaCas9 DNA to blood cells. FIG. 12A depicts representative quantification of SaCas9 DNA in blood cells isolated from MISTRG-6-15 mice treated with AAV-SaCas9/PD-1 (g1+g3). FIG. 12B depicts representative quantification of cells by species found in MISTRG-6-15 mice.

FIG. 13, comprising FIG. 13A and FIG. 13B, depicts representative in vivo delivery of SaCas9 DNA to blood cells. FIG. 13A depicts representative quantification of SaCas9 DNA in blood cells isolated from MISTRG-6-15 mice treated with AAV6-SaCas9/PD-1 (g1+g3). FIG. 13B depicts representative quantification of cells by species found in MISTRG-6-15 mice before and after enrichment for human cells.

FIG. 14, comprising FIG. 14A and FIG. 14B, depicts representative ddPCR quantification of AAV DNA levels in MISTRG-6-15 tissues. FIG. 14A depicts representative images of ddPCR droplet distribution. Each droplet in a sample is plotted on a graph of fluorescence intensity versus droplet number. All positive droplets (those above the threshold intensity line) are scored as positive, and each is assigned a value of 1. All negative droplets (those below the threshold) are scored as negative and assigned a value of 0. FIG. 14B depicts representative quantification of SaCas9 DNA copy number per cell in spleen and liver tissue. Copy number per cell was calculated according to

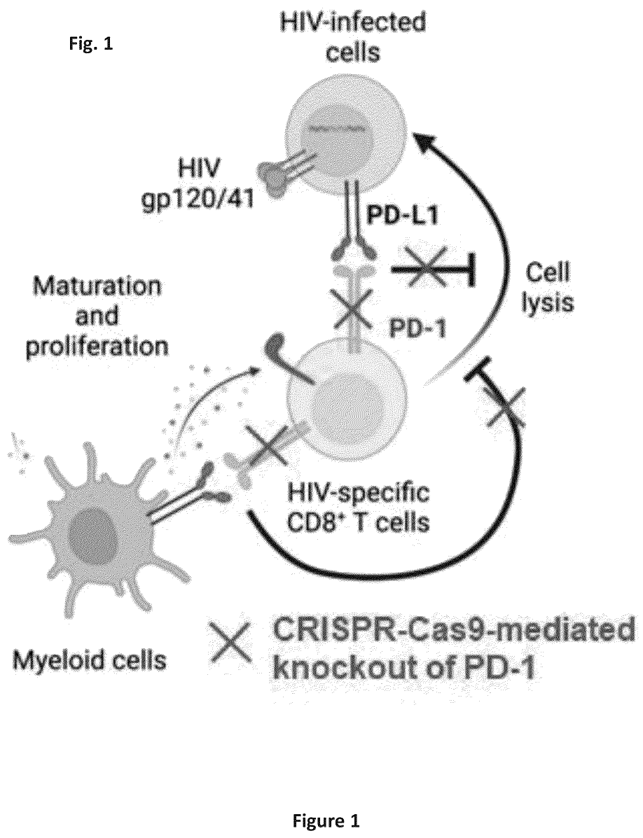

$$Copy per Cell = \frac{Copy per Well}{Human copy per well + Mouse copy per well} \times 2 .$$

### DESCRIPTION OF THE INVENTION

The invention is based, in part, on the finding that silencing of programed cell death protein 1 (PD-1) rescues cytotoxic T lymphocyte (CTL) exhaustion. The invention is further based on the finding that a vector encoding a Staphylococcus aureus 9 (SaCas9) and PD-1 guide molecules can down-regulate PD-1 expression in T cells.

Therefore, in various embodiments, the invention relates to a genome editing system comprising a vector encoding a SaCas9 and at least one gRNA molecule comprising a targeting domain that is complementary with a target sequence of a PD-1 gene.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of 20%, ±10%, ±5%, ±1%, or ±0.1% from the specified value, as such variations are appropriate to perform the disclosed methods.

The term “abnormal” when used in the context of organisms, tissues, cells, or components thereof, refers to those organisms, tissues, cells, or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

The term “activate,” as used herein, means to induce or increase an activity or function, for example, about ten percent relative to a control value. Preferably, the activity is induced or increased by 50% compared to a control value, more preferably by 75%, and even more preferably by 95%. “Activate,” as used herein, also means to increase a molecule, a reaction, an interaction, a gene, an mRNA, and/or a protein's expression, stability, function, or activity by a measurable amount or to increase entirely. Activators are compounds that, e.g., bind to, partially or totally induce stimulation, increase, promote, induce activation, activate, sensitize, or upregulate a protein, a gene, and an mRNA stability, expression, function, and activity, e.g., agonists.

“Antisense” refers particularly to the nucleic acid sequence of the non-coding strand of a double stranded DNA molecule, or to a sequence which is substantially homologous to the non-coding strand. As defined herein, an antisense sequence is complementary to the sequence of a double stranded DNA molecule. It is not necessary that the antisense sequence be complementary solely to the coding portion of the coding strand of the DNA molecule. The antisense sequence may be complementary to regulatory sequences specified on the coding strand of a DNA molecule, which regulatory sequences control expression of the coding sequences.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a sign or symptom of the disease or disorder, the frequency with which such a sign or symptom is experienced by a patient, or both, is reduced.

An “effective amount” or “therapeutically effective amount” of a compound is that amount of a compound which is sufficient to provide a beneficial effect to the subject to which the compound is administered. An “effective amount” of a delivery vehicle is that amount sufficient to effectively bind or deliver a compound.

The term “inhibit,” as used herein, means to suppress or block an activity or function, for example, about ten percent relative to a control value. Preferably, the activity is suppressed or blocked by 50% compared to a control value, more preferably by 75%, and even more preferably by 95%. “Inhibit,” as used herein, also means to reduce a molecule, a reaction, an interaction, a gene, an mRNA, and/or a protein's expression, stability, function, or activity by a measurable amount or to prevent entirely. Inhibitors are compounds that, e.g., bind to, partially or totally block stimulation, decrease, prevent, delay activation, inactivate, desensitize, or down regulate a protein, a gene, and an mRNA stability, expression, function, and activity, e.g., antagonists.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

As used herein, a “modulator of PD-1” is a compound that modifies the expression, activity or biological function of PD-1 protein or RNA as compared to the expression, activity or biological function of the PD-1 protein or RNA in the absence of the modulator.

The terms “patient,” “subject,” “individual,” and the like are used interchangeably herein, and refer to any animal, or cells thereof whether in vitro or in vivo, amenable to the methods described herein. In certain non-limiting embodiments, the patient, subject or individual is a human.

A “therapeutic” treatment is a treatment administered to a subject who exhibits signs or symptoms of a disease or disorder, for the purpose of diminishing or eliminating those signs or symptoms.

As used herein, “treating a disease or disorder” means reducing the severity and/or frequency with which a sign or symptom of the disease or disorder is experienced by a patient.

The phrase “biological sample” as used herein, is intended to include any sample comprising a cell, a tissue, or a bodily fluid in which expression of a nucleic acid or polypeptide is present or can be detected. Samples that are liquid in nature are referred to herein as “bodily fluids.” Biological samples may be obtained from a patient by a variety of techniques including, for example, by scraping or swabbing an area of the subject or by using a needle to obtain bodily fluids. Methods for collecting various body samples are well known in the art.

As used herein, an “immunoassay” refers to any binding assay that uses an antibody capable of binding specifically to a target molecule to detect and quantify the target molecule.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from at least one species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific.

In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A,” the presence of a molecule containing epitope A (or free, unlabeled A), in a reaction containing labeled “A” and the antibody, will reduce the amount of labeled A bound to the antibody.

A “coding region” of a gene consists of the nucleotide residues of the coding strand of the gene and the nucleotides of the non-coding strand of the gene.

“Complementary” as used herein to refer to a nucleic acid, refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

“Isolated” means altered or removed from the natural state. For example, a nucleic acid or a peptide naturally present in its normal context in a living animal is not “isolated,” but the same nucleic acid or peptide partially or completely separated from the coexisting materials of its natural context is “isolated.” An isolated nucleic acid or protein can exist in substantially purified form, or can exist in a non-native environment such as, for example, a host cell.

An “isolated nucleic acid” refers to a nucleic acid segment or fragment which has been separated from sequences which flank it in a naturally occurring state, i.e., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, i.e., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes a recombinant DNA which is part of a hybrid gene encoding additional polypeptide sequence.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention relates generally to compositions and methods for modulating PD-1. In some embodiments, the compositions of the present invention comprise a modulator of the activity or expression of PD-1. In some embodiments, the composition comprises a vector that alters or deletes all or a portion of a gene encoding PD-1.

In some embodiments, the compositions and methods are directed to preventing or treating a retroviral infection. In some embodiments, the retroviral infection is a human immunodeficiency virus (HIV) infection.

### Compositions

In various embodiments, the present invention provides compositions for modulating the level or activity of PD-1 in a subject, a cell, a tissue, or an organ in need thereof. In some embodiments, the composition modulates the expression of a gene encoding PD-1, the half-life of an mRNA molecule encoding PD-1, the translation of an mRNA molecule encoding PD-1, the half-life of a PD-1 protein, and/or the activity of a PD-1 protein. In some embodiments, the modulator modifies, disrupts, or delete all of or a portion of a gene encoding PD-1. That is, the routineer would appreciate, based upon the disclosure provided herein, that modulating the level or activity of a gene, or gene product, can be readily assessed using methods that assess the level of a nucleic acid encoding a gene product (e.g., mRNA, lncRNA), the level of gene product present in a biological sample, the activity of gene product present in a biological sample, or combinations thereof.

The modulator compositions of the invention that modulate the level or activity of a gene, or gene product, include, but should not be construed as being limited to, a chemical compound, a protein, a peptide, a peptidomimetic, an antibody, a ribozyme, a small molecule chemical compound, a nucleic acid, a vector, an antisense nucleic acid molecule (e.g., siRNA, miRNA, etc.), or combinations thereof. One of skill in the art would readily appreciate, based on the disclosure provided herein, that a modulator composition encompasses a chemical compound that modulates the level or activity of a gene, or gene product. Additionally, a modulator composition encompasses a chemically modified compound, and derivatives, as is well known to one of skill in the chemical arts.

Further, one of skill in the art would, when equipped with this disclosure and the methods exemplified herein, appreciate that modulators include such modulators as discovered in the future, as can be identified by well-known criteria in the art of pharmacology, such as the physiological results of modulation of the genes, and gene products, as described in detail herein and/or as known in the art. Therefore, the present invention is not limited in any way to any particular modulator composition as exemplified or disclosed herein; rather, the invention encompasses those modulator compositions that would be understood by the routineer to be useful as are known in the art and as are discovered in the future.

Further methods of identifying and producing modulator compositions are well known to those of ordinary skill in the art. Alternatively, a modulator can be synthesized chemically. Further, the person of skill in the art would appreciate, based upon the teachings provided herein, that a modulator composition can be obtained from a recombinant organism. Compositions and methods for chemically synthesizing modulators and for obtaining them from natural sources are well known in the art and are described in the art.

One of skill in the art will appreciate that a modulator can be administered as a small molecule chemical, a polypeptide, a peptide, an antibody, a nucleic acid construct encoding a protein, an antisense nucleic acid (e.g., siRNA, miRNA, etc.), a nucleic acid construct encoding an antisense nucleic acid, or combinations thereof. Numerous vectors and other compositions and methods are well known for administering a small molecule chemical, a polypeptide, a peptide, an antibody, a nucleic acid construct encoding a protein, an antisense nucleic acid (e.g., siRNA, miRNA, etc.), or a nucleic acid construct encoding an antisense nucleic acid to cells or tissues.

In some embodiments, the invention provides a generic concept for inhibiting PD-1. In some embodiments, the inhibitor is selected from the group consisting of a small interfering RNA (siRNA), shRNA, a microRNA, a guide RNA, an antisense nucleic acid, a ribozyme, an expression vector encoding a transdominant negative mutant, an intracellular antibody, a peptide, a small molecule, and combinations thereof.

### Nucleic Acids

In some embodiments, the composition of the invention comprises at least one antisense nucleic acid molecules. For example, in some embodiments, the at least one antisense nucleic acid molecules are specific for targeting PD-1 mRNA. Antisense oligonucleotides are DNA or RNA molecules that are complementary to some portion of a mRNA or lncRNA molecule. When present in a cell, antisense oligonucleotides hybridize to an existing mRNA or lncRNA molecule and inhibit translation into a gene product or promote degradation of the RNA molecule. Inhibiting the expression of a gene using an antisense oligonucleotide is well known in the art (Marcus-Sekura, 1988, Anal. Biochem. 172:289), as are methods of expressing an antisense oligonucleotide in a cell (Inoue, U.S. Pat. No. 5,190,931). The methods of the invention include the use of antisense oligonucleotide to diminish the amount of PD-1 activity or PD-1 mRNA. Contemplated in the present invention are antisense oligonucleotides that are synthesized and provided to the cell by way of methods well known to those of ordinary skill in the art. As an example, an antisense oligonucleotide can be synthesized to be between about 10 and about 100, more preferably between about 15 and about 50 nucleotides long. The synthesis of nucleic acid molecules is well known in the art, as is the synthesis of modified antisense oligonucleotides to improve biological activity in comparison to unmodified antisense oligonucleotides (Tullis, 1991, U.S. Pat. No. 5,023,243).

Similarly, the expression of a gene may be inhibited by the hybridization of an antisense molecule to a promoter or other regulatory element of a gene, thereby affecting the transcription of the gene. Methods for the identification of a promoter or other regulatory element that interacts with a gene of interest are well known in the art, and include such methods as the yeast two hybrid system (Bartel and Fields, eds., In: The Yeast Two Hybrid System, Oxford University Press, Cary, N.C.).

Alternatively, inhibition of PD-1 can be accomplished through the use of an siRNA, shRNA, antisense oligonucleotide, or ribozyme. Given the nucleotide sequence of the molecule, one of ordinary skill in the art could synthesize an antisense oligonucleotide or ribozyme without undue experimentation, provided with the disclosure and references incorporated herein.

In some embodiments, siRNA is used to decrease the level of PD-1. RNA interference (RNAi) is a phenomenon in which the introduction of double-stranded RNA (dsRNA) into a diverse range of organisms and cell types causes degradation of the complementary mRNA. In the cell, long dsRNAs are cleaved into short 21-25 nucleotide small interfering RNAs, or siRNAs, by a ribonuclease known as Dicer. The siRNAs subsequently assemble with protein components into an RNA-induced silencing complex (RISC), unwinding in the process. Activated RISC then binds to complementary transcript by base pairing interactions between the siRNA antisense strand and the mRNA. The bound mRNA is cleaved and sequence specific degradation of mRNA results in gene silencing. See, for example, U.S. Pat. No. 6,506,559; Fire et al., 1998, Nature 391(19):306-311; Timmons et al., 1998, Nature 395:854; Montgomery et al., 1998, TIG 14 (7):255-258; David R. Engelke, Ed., RNA Interference (RNAi) Nuts & Bolts of RNAi Technology, DNA Press, Eagleville, PA (2003); and Gregory J. Hannon, Ed., RNAi A Guide to Gene Silencing, Cold Spring Harbor Laboratory Press, Cold Spring Harbor, NY (2003). Soutschek et al. (2004, Nature 432:173-178) describe a chemical modification to siRNAs that aids in systemic delivery. Optimizing siRNAs involves consideration of overall G/C content, C/T content at the termini, Tm, and the nucleotide content of the 3′ overhang. See, for instance, Schwartz et al., 2003, Cell, 115:199-208 and Khvorova et al., 2003, Cell 115:209-216. Therefore, the present invention also includes methods of decreasing levels of at least one PD-1 at the protein level using RNAi technology.

In certain embodiments, the modulators described herein comprise short hairpin RNA (shRNA) molecules. shRNA molecules are well known in the art and are directed against the mRNA of a target, thereby decreasing the expression of the target. In certain embodiments, the encoded shRNA is expressed by a cell, and is then processed into siRNA. For example, in certain instances, the cell possesses native enzymes (e.g., dicer) that cleaves the shRNA to form siRNA.

In some embodiments, the inhibitor of the invention is an antisense molecule. Antisense molecules of the invention may be made synthetically and then provided to the cell. Antisense oligomers of between about 10 to about 30, and more preferably about 15 nucleotides, are preferred since they are easily synthesized and introduced into a target cell. Synthetic antisense molecules contemplated by the invention include oligonucleotide derivatives known in the art which have improved biological activity compared to unmodified oligonucleotides (see U.S. Pat. No. 5,023,243).

Ribozymes and their use for inhibiting gene expression are also well known in the art (see, e.g., Cech et al., 1992, J. Biol. Chem. 267:17479-17482; Hampel et al., 1989, Biochemistry 28:4929-4933; Eckstein et al., International Publication No. WO 92/07065; Altman et al., U.S. Pat. No. 5,168,053). Ribozymes are RNA molecules possessing the ability to specifically cleave other single-stranded RNA in a manner analogous to DNA restriction endonucleases. Through the modification of nucleotide sequences encoding these RNAs, molecules can be engineered to recognize specific nucleotide sequences in an RNA molecule and cleave it (Cech, 1988, J. Amer. Med. Assn. 260:3030). A major advantage of this approach is the fact that ribozymes are sequence-specific.

There are two basic types of ribozymes, namely, tetrahymena-type (Hasselhoff, 1988, Nature 334:585) and hammerhead-type. Tetrahymena-type ribozymes recognize sequences which are four bases in length, while hammerhead-type ribozymes recognize base sequences 11-18 bases in length. The longer the sequence, the greater the likelihood that the sequence will occur exclusively in the target mRNA species. Consequently, hammerhead-type ribozymes are preferable to tetrahymena-type ribozymes for inactivating specific mRNA species, and 18-base recognition sequences are preferable to shorter recognition sequences which may occur randomly within various unrelated mRNA molecules.

In some embodiments of the invention, a ribozyme is used to inhibit PD-1. Ribozymes useful for inhibiting the expression of a target molecule may be designed by incorporating target sequences into the basic ribozyme structure which are complementary, for example, to the mRNA sequence of PD-1 of the present invention. Ribozymes targeting at PD-1 may be synthesized using commercially available reagents or they may be genetically expressed from DNA encoding them.

In some embodiments, the inhibitor of PD-1 may comprise at least one components of a CRISPR-Cas system, where a guide RNA (gRNA) targeted to a gene encoding PD-1 and a CRISPR-associated (Cas) peptide form a complex to induce mutations within the targeted gene. In some embodiments, the inhibitor comprises a gRNA or a nucleic acid molecule encoding a gRNA.

In some embodiments, the at least one gRNA molecule comprises a targeting domain that is complementary to a target sequence of a PD-1 gene. In some embodiments, the PD1-gene is a human PD-1 gene. In some embodiments, the gRNA comprises a nucleotide sequence having at least about 50% identity, at least about 60% identity, at least about 70% identity, at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, or at least about 99% identity to a nucleotide sequence selected from the group consisting of SEQ ID NOs:1-5. In some embodiments, the gRNA molecules comprises a nucleotide sequence selected from the group consisting of SEQ ID NOs:1-5.

In some embodiments, the inhibitor of PD-1 comprises at least two gRNA molecules, at least one nucleic acid molecule encoding at least two gRNA molecules, or at least two nucleic acid molecules encoding at least two gRNA molecules. In some embodiments, the at least two gRNA molecules comprise at least two sequences having at least about 50% identity, at least about 60% identity, at least about 70% identity, at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, or at least about 99% identity to at least two nucleotide sequences selected from the group consisting of SEQ ID NOs:1-5. In some embodiments, the at least two gRNA molecules comprises at least two nucleotide sequences selected from the group consisting of SEQ ID NOs:1-5.

In some embodiments, the first of the at least two gRNA molecules comprises the nucleotide sequence of SEQ ID NO:1 and the second of the at least two gRNA molecules comprises the nucleotide sequence of SEQ ID NO:2. In some embodiments, the first of the at least two gRNA molecules comprises the nucleotide sequence of SEQ ID NO:1 and the second of the at least two gRNA molecules comprises the nucleotide sequence of SEQ ID NO:3. In some embodiments, the first of the at least two gRNA molecules comprises the nucleotide sequence of SEQ ID NO:1 and the second of the at least two gRNA molecules comprises the nucleotide sequence of SEQ ID NO:4. In some embodiments, the first of the at least two gRNA molecules comprises the nucleotide sequence of SEQ ID NO:1 and the second of the at least two gRNA molecules comprises the nucleotide sequence of SEQ ID NO:5.

In some embodiments, the first of the at least two gRNA molecules comprises the nucleotide sequence of SEQ ID NO:2 and the second of the at least two gRNA molecules comprises the nucleotide sequence of SEQ ID NO:3. In some embodiments, the first of the at least two gRNA molecules comprises the nucleotide sequence of SEQ ID NO:2 and the second of the at least two gRNA molecules comprises the nucleotide sequence of SEQ ID NO:4. In some embodiments, the first of the at least two gRNA molecules comprises the nucleotide sequence of SEQ ID NO:2 and the second of the at least two gRNA molecules comprises the nucleotide sequence of SEQ ID NO:5.

In some embodiments, the first of the at least two gRNA molecules comprises the nucleotide sequence of SEQ ID NO:3 and the second of the at least two gRNA molecules comprises the nucleotide sequence of SEQ ID NO:4. In some embodiments, the first of the at least two gRNA molecules comprises the nucleotide sequence of SEQ ID NO:3 and the second of the at least two gRNA molecules comprises the nucleotide sequence of SEQ ID NO:5.

In some embodiments, the first of the at least two gRNA molecules comprises the nucleotide sequence of SEQ ID NO:4 and the second of the at least two gRNA molecules comprises the nucleotide sequence of SEQ ID NO:5.

In some embodiments, the inhibitor comprises a Cas peptide or a nucleic acid molecule encoding a Cas peptide. In some embodiments, the Cas peptide is a Cas9 peptide. In some embodiments, the Cas9 peptide is a Staphylococcus aureus Cas9 (SaCas9) peptide.

In some embodiments, the inhibitor comprises at least one gRNA molecule or a nucleic acid molecule encoding at least one gRNA molecules and a Cas peptide or a nucleic acid molecule encoding a Cas peptide. In some embodiments, the inhibitor comprises at least two gRNA molecules, at least one nucleic acid molecule encoding at least two gRNA molecules, or at least two nucleic acid molecules encoding at least two gRNA molecules and a Cas peptide or a nucleic acid molecule encoding a Cas peptide.

In some embodiments, a nucleic acid modulator comprises at least one nucleotide modification. In some embodiments, the modifications are at least one of 2′-O-methyl, 2′-O-fluoro, and phosphorothioate. In certain embodiments, the nucleotide is modified at the 2′ position of the sugar moiety. In certain embodiments, the modification at the 2′ position of the sugar moiety is 2′-O-methyl or 2′-O-fluoro. In certain embodiments, the nucleotide is modified at the 3′ position of the sugar moiety. In certain embodiments, the modification at the 3′ position of the sugar moiety is phosphorothioate. In certain embodiments, the nucleotide is modified at both the 2′ position of the sugar moiety and at the 3′ position of the sugar moiety. In certain embodiments, the nucleotide is not modified at the 2′ position of the sugar moiety. In certain embodiments, the nucleotide is not modified at the 3′ position of the sugar moiety.

In some embodiments, the nucleotide modification is at least one locked nucleic acid (LNA) connecting adjacent nucleotides. Other modifications include but are not limited to, 2′-modified RNA phosphoramidites (e.g., 2′-OMe), 2′-methoxy (2′-OCH3), 2′-aminopropoxy (2′-OCH2CH2CH2NH2), 2′-O-methoxyethyl (2MOE), and 2′-fluoro (2′-F). Modifications may be made at any position on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar.

Nucleic acid molecules may also include a modified thioester group on the 2′, 3′ and/or 5′ nucleoside. Such modifications in the 5′ carbon of the ribose sugar also for formation of single 5′-S-thioester linkages between nucleotides in a synthetic nucleotide sequence. In any 3′ or 5′ linkage between nucleotides any one or both positions may create a series of linkages between nucleotides. The linkages at the 2′ or 3′ can create thioester bond, phosphorothioriate linkages between two or a plurality of nucleosides in the oligonucleotide.

Strategically placed sulfur atoms in the backbone of nucleic acids have found widespread utility in probing of specific interactions of proteins, enzymes, and metals. In some embodiments, sulfur replacement for oxygen may be carried out at the 2′-position of RNA and in the 3′-5′-positions of RNA and of DNA. In some embodiments, linkers of any cyclic or acyclic hydrocarbon chains of varying length may be incorporated into the nucleic acid. In some embodiments, linkers of the disclosure comprise one or a plurality of: branched or non-branched alkyl, hydroakyl, hydroxyl, halogen, metal, nitrogen, or other atoms.

Nucleic acid molecules may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Certain of these nucleobases are particularly useful for increasing the binding affinity of the nucleic acid molecules of the disclosure. These include 5-substituted pyrimidines, 6-azapyrimidines and N-2, N-6 and 0-6 substituted purines, including 2-aminopropyladenine, 5-propynyluracil and 5-propynylcytosine.

In some embodiments, the inhibitor comprises a promoter/regulatory sequence such that the nucleic acid is capable of directing expression of the nucleic acid.

### Vectors

In some embodiments, the invention relates to a vector, comprising at least one nucleic acid inhibitor of the invention. The choice of the vector will depend on the host cell in which it is to be subsequently introduced. In a particular embodiment, the vector of the invention is an expression vector. In some embodiments, the vector comprises a plasmid.

In some embodiments, the plasmid comprises a nucleic acid sequence encoding a Cas peptide and at least one nucleic acid sequence encoding at least one gRNA molecule. In some embodiments, the Cas peptide is a Cas9 peptide. In some embodiments, the Cas9 peptide is a Staphylococcus aureus Cas9 (SaCas9) peptide. In some embodiments, the SaCas9 peptide is an adeno-associated virus (AAV)-compatible SaCas9.

In some embodiments, the plasmid comprises at least one nucleic acid sequence encoding at least one gRNA molecule, wherein the at least one gRNA molecule comprises a nucleotide sequence having at least about 50% identity, at least about 60% identity, at least about 70% identity, at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, or at least about 99% identity to a nucleotide sequence selected from the group consisting of SEQ ID NOs:1-5. In some embodiments, the at least one gRNA molecules comprises a nucleotide sequence selected from the group consisting of SEQ ID NOs:1-5.

In some embodiments, the plasmid comprises at least two nucleic acid sequences encoding at least two gRNA molecules, wherein the at least two sequences having at least about 50% identity, at least about 60% identity, at least about 70% identity, at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, or at least about 99% identity to two nucleotide sequences selected from the group consisting of SEQ ID NOs:1-5. In some embodiments, the at least two gRNA molecules comprises at least two nucleotide sequences selected from the group consisting of SEQ ID NOs:1-5.

The vector may be obtained by conventional methods known by persons skilled in the art (Sambrook et al., 2012). In a particular embodiment, the vector is a vector useful for transforming animal cells. In one embodiment, the recombinant expression vectors may also contain nucleic acid molecules which encode a nucleic acid inhibitor of invention, described elsewhere herein.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (U.S. Pat. Nos. 4,683,202, 5,928,906). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know how to use promoters, enhancers, and cell type combinations for protein expression, for example, see Sambrook et al. (2012). The promoters employed may be constitutive, tissue-specific, inducible, and/or useful under the appropriate conditions to direct high-level expression of the introduced DNA segment, such as is advantageous in the large-scale production of recombinant proteins and/or peptides. The promoter may be heterologous or endogenous.

In some embodiments, the vector is an expression vector for cell-specific or tissue-specific expression. In some embodiments, the vector is a T cell-specific vector.

Further, the expression vector may be provided in the form of a viral vector. Viral vector technology is well known in the art and is described, for example, in Sambrook et al. (2012), and in Ausubel et al. (1997), and in other virology and molecular biology manuals. Viruses, which are useful as vectors include, but are not limited to, retroviruses, adenoviruses, adeno-associated viruses (AAVs), herpes viruses, and lentiviruses. In general, a suitable vector contains an origin of replication functional in at least one organism, a promoter sequence, convenient restriction endonuclease sites, and one or more selectable markers. (See, e.g., WO 01/96584; WO 01/29058; and U.S. Pat. No. 6,326,193. In some embodiments, the viral vector is an AAV. Exemplary AAV serotypes include, but is not limited to AAV1, AAV2, AAV3, AAV4, AAV5, AAV6, AAV7, AAV8 and AAV9. In some embodiments, the AAV is an AAV6 or AAV9 serotype.

In some embodiments, the vector is a viral vector comprising a plasmid of the present invention. In some embodiments, the viral vector is an AAV. In some embodiments, the AAV is an AAV6 or AAV9 serotype.

### Pharmaceutical Compositions

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the description of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

Pharmaceutical compositions that are useful in the methods of the invention may be prepared, packaged, or sold in formulations suitable for intrathecal, ophthalmic, oral, rectal, vaginal, parenteral, topical, pulmonary, intranasal, buccal, intratumoral, or another route of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents, including, for example, chemotherapeutics, immunosuppressants, corticosteroids, analgesics, and the like.

Controlled- or sustained-release formulations of a pharmaceutical composition of the invention may be made using conventional technology.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intrathecal, intraocular, intravitreal, subcutaneous, intraperitoneal, intramuscular, intrasternal injection, intratumoral, and kidney dialytic infusion techniques. In one embodiment, the method of administration is through intrathecal injection.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multidose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The disclosure also relates to pharmaceutical compositions comprising: (i) an inhibitor of PD-1 of the present invention; and (ii) a pharmaceutically acceptable carrier. The term “pharmaceutically acceptable salts” refers to physiologically and pharmaceutically acceptable salts of the nucleic acid sequences of the disclosure: i.e., salts that retain the desired biological activity of the nucleic acid sequences and do not impart undesired toxicological effects thereto.

Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine (see, for example, Berge et al., “Pharmaceutical Salts,” J. of Pharnut Sci., 1977, 66:1). The base addition salts of said acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present disclosure. As used herein, a “pharmaceutical addition salt” includes a pharmaceutically acceptable salt of an acid form of one of the components of the compositions of the disclosure. These include organic or inorganic acid salts of the amines. In some embodiments, a pharmaceutically acceptable salt is selected from one or a combination of hydrochlorides, acetates, salicylates, nitrates and phosphates.

Other suitable pharmaceutically acceptable salts are well known to those skilled in the art and include basic salts of a variety of inorganic and organic acids, such as, for example, with inorganic acids, such as for example hydrochloric acid, hydrobromic acid, sulfuric acid or phosphoric acid; with organic carboxylic, sulfonic, sulfo or phospho acids or N-substituted sulfamic acids; for example acetic acid, propionic acid, glycolic acid, succinic acid, malefic acid, hydroxymaleic acid, methylmaleic acid, fumaric acid, malic acid, tartaric acid, lactic acid, oxalic acid, gluconic acid, glucaric acid, glucuronic acid, citric acid, benzoic acid, cinnamic acid, mandelic acid, salicylic acid, 4-aminosalicylic acid, 2phenoxybenzoic acid, 2-acetoxybenzoic acid, embonic acid, nicotinic acid or isonicotinic acid; and with amino acids, such as the 20 alpha-amino acids involved in the synthesis of proteins in nature, for example glutamic acid or aspartic acid, and also with phenylacetic acid, methanesulfonic acid, ethanesulfonic acid, 2-hydroxyethanesulfonic acid, ethane-1,2-disulfonic acid, benzenesulfonic acid, 4-methylbenzenesulfonic acid, naphthalene-2-sulfonic acid, naphthalene-1,5-disulfonic acid, 2- or 3-phosphoglycerate, glucose-6phosphate, N-cyclohexylsulfamic acid (with the formation of cyclamates), or with other acid organic compounds, such as ascorbic acid. Pharmaceutically acceptable salts of compounds may also be prepared with a pharmaceutically acceptable cation. Suitable pharmaceutically acceptable cations are well known to those skilled in the art and include alkaline, alkaline earth, ammonium and quaternary ammonium cations. Carbonates or hydrogen carbonates are also possible.

For oligonucleotides, examples of pharmaceutically acceptable salts include but are not limited to (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, malefic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multidose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations that are useful include those that comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in the inventive formulations. Excipients such as cocoa butter and suppository waxes, coloring agents, coating agents, sweetening, flavoring, and perfuming agents can be present in the composition, according to the judgment of the formulator.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include, but are not limited to, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof.

Exemplary binding agents include, but are not limited to, starch (e.g. cornstarch and starch paste); gelatin; sugars (e.g. sucrose, glucose, dextrose, dextrin, molasses, lactose, lactitol, mannitol); natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan); alginates; polyethylene oxide; polyethylene glycol; inorganic calcium salts; silicic acid; polymethacrylates; waxes; water; alcohol; etc.; and combinations thereof.

Additionally, the molecules may be delivered using a sustained-release system, such as semipermeable matrices of solid polymers containing the therapeutic agent. Various forms of sustained-release materials have been established and are well known by those skilled in the art. Sustained-release capsules may, depending on their chemical nature, release the molecules for a few weeks up to over 100 days. Depending on the chemical nature and the biological stability of the chimeric molecules, additional strategies for molecule stabilization may be employed.

Nucleic acids may be included in any of the above-described formulations as the free acids or bases or as pharmaceutically acceptable salts. Pharmaceutically acceptable salts are those salts that substantially retain the biologic activity of the free bases and which are prepared by reaction with inorganic acids. Pharmaceutical salts tend to be more soluble in aqueous and other protic solvents than are the corresponding free base forms.

In addition to the formulations described previously, the molecules may also be formulated as a depot preparation. Thus, the molecules may be formulated with suitable polymeric or hydrophobic materials (for example as an emulsion in an acceptable oil) or ion exchange resins, or as sparingly soluble derivatives, for example, as a sparingly soluble salt.

In some embodiments, the composition or pharmaceutical composition comprises any nucleic acid disclosed herein or its salt and one or more additional therapies. In some embodiments, the pharmaceutical composition comprises any one or plurality of nucleic acids disclosed herein or its salt or variant thereof and/or one or more therapies is administered to the subject before, contemporaneously with, substantially contemporaneously with, or after administration of the pharmaceutical composition.

Compositions of the disclosure include pharmaceutical compositions comprising: a particle comprising any of the nucleic acid sequences disclosed herein, or pharmaceutically acceptable salts thereof, and a pharmaceutically acceptable carrier. In some embodiments, the pharmaceutically acceptable carrier is distilled water or saline. In preferred embodiments, the pharmaceutically acceptable carrier is free of RNase/DNase.

As used herein, a “particle” refers to any entity having a diameter of less than 100 microns (μm). Typically, particles have a longest dimension (e.g. diameter) of 1000 nm or less. In some embodiments, particles have a diameter of 300 nm or less. In some embodiments, nanoparticles have a diameter of 200 nm or less. In some embodiments, nanoparticles have a diameter of 100 nm or less. In general, particles are greater in size than the renal excretion limit, but are small enough to avoid accumulation in the liver. In some embodiments, a population of particles may be relatively uniform in terms of size, shape, and/or composition. In general, inventive particles are biodegradable and/or biocompatible. Inventive particles can be solid or hollow and can comprise one or more layers. In some embodiments, particles are spheres, spheroids, flat, plate-shaped, cubes, cuboids, ovals, ellipses, cylinders, cones, or pyramids. In some embodiments, particles can be a matrix of polymers. In some embodiments, the matrix is cross-linked. In some embodiments, formation of the matrix involves a cross-linking step. In some embodiments, the matrix is not substantially cross-linked. In some embodiments, formation of the matrix does not involve a cross-linking step. In some embodiments, particles can be a non-polymeric particle (e.g. a metal particle, quantum dot, ceramic, inorganic material, bone, etc.). Components of the pharmaceutical compositions disclosed herein may comprise particles or may be microparticles, nanoparticles, liposomes, and/or micelles comprising one or more disclosed nucleic acid sequences or conjugated to one or more disclosed nucleic acids. As used herein, the term “nanoparticle” refers to any particle having a diameter of less than 1000 nm. In some embodiments, the particle is an exosome.

The practice of the present disclosure employs, unless otherwise indicated, conventional techniques of immunology, biochemistry, chemistry, molecular biology, microbiology, cell biology, genomics and recombinant DNA, which are within the skill of the art.

### Therapeutic Methods

The disclosure also provides methods of modulating or inhibiting expression of PD-1 in a subject. In some embodiments, the method comprises selectively modulating or inhibiting expression of PD-1 in a specific cell or tissue type of a subject. In some embodiments, the method comprises administering to a subject a PD-1 inhibitor of the present invention. In some embodiments, the method comprises modulating or inhibiting expression of PD-1 in a target cell type of a subject comprising contacting the cell type with an inhibitor of the present invention. In some embodiments, the method comprises altering a target cell type. In some embodiments, the method comprises disrupting, altering, or deleting all or a portion of a PD-1 gene in the target cell type. In some embodiments, the target cell type is a T cell. In some embodiments, the T cell is a CD8⁺ T cell. In some embodiments, the T cell is a cytotoxic T lymphocyte (CTL).

In some embodiments, the method comprises contacting a target cell with a genome editing system. In some embodiments, the genome editing system comprises a Cas peptide or a nucleic acid molecule encoding a Cas peptide and at least one gRNA molecule comprising a targeting domain that is complementary to a target sequence of a PD-1 gene or a nucleic acid molecule encoding at least one gRNA. In some embodiments, the Cas peptide is a Cas9 peptide. In some embodiments, the Cas9 peptide is a Staphylococcus aureus Cas9 (SaCas9) peptide. In some embodiments, the SaCas9 peptide is an adeno-associated virus (AAV)-compatible SaCas9.

In some embodiments, the at least one gRNA molecule comprises a nucleic acid sequence having at least about 50% identity, at least about 60% identity, at least about 70% identity, at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, or at least about 99% identity to a nucleotide sequence selected from the group consisting of SEQ ID NOs:1-5. In some embodiments, the gRNA molecules comprises a nucleotide sequence selected from the group consisting of SEQ ID NOs:1-5.

In some embodiments, the genome editing system comprises a Cas peptide or a nucleic acid molecule encoding a Cas peptide and at least two gRNA molecules comprising at least two targeting domains that are complementary to at least two target sequences of a PD-1 gene, at least one nucleic acid molecule encoding at least two gRNA molecules, or at least two nucleic acid molecules encoding at least two gRNA molecules. In some embodiments, the at least two gRNA molecules comprise at least two sequences having at least about 50% identity, at least about 60% identity, at least about 70% identity, at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, or at least about 99% identity to at least two nucleotide sequences selected from the group consisting of SEQ ID NOs:1-5. In some embodiments, the at least two gRNA molecules comprises at least two nucleotide sequences selected from the group consisting of SEQ ID NOs:1-5.

In some embodiments, the method comprises contacting a target cell with a plasmid. In some embodiments, the plasmid encodes a genome editing system. In some embodiments, the plasmid comprises a nucleic acid sequence encoding a Cas peptide and at least one nucleic acid sequence encoding at least one gRNA molecule comprising a targeting domain that is complementary to a target sequence of a PD-1 gene. In some embodiments, the Cas peptide is a Cas9 peptide. In some embodiments, the Cas9 peptide is a SaCas9 peptide. In some embodiments, the SaCas9 peptide is an AAV-compatible SaCas9. In some embodiments, the at least one gRNA molecule comprises a nucleic acid sequence having at least about 50% identity, at least about 60% identity, at least about 70% identity, at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, or at least about 99% identity to a nucleotide sequence selected from the group consisting of SEQ ID NOs:1-5. In some embodiments, the gRNA molecules comprises a nucleotide sequence selected from the group consisting of SEQ ID NOs:1-5.

In some embodiments, the plasmid comprises a nucleic acid sequence encoding a Cas peptide and at least two nucleic acid sequences encoding at least two gRNA molecules comprising at least two targeting domains that are complementary to at least two target sequences of a PD-1 gene. In some embodiments, the Cas peptide is a Cas9 peptide. In some embodiments, the Cas9 peptide is a SaCas9 peptide. In some embodiments, the SaCas9 peptide is an AAV-compatible SaCas9. In some embodiments, the at least two gRNA molecules comprise at least two sequences having at least about 50% identity, at least about 60% identity, at least about 70% identity, at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, or at least about 99% identity to at least two nucleotide sequences selected from the group consisting of SEQ ID NOs:1-5. In some embodiments, the at least two gRNA molecules comprises at least two nucleotide sequences selected from the group consisting of SEQ ID NOs:1-5.

In some embodiments, the method comprises contacting a target cell with a viral vector. In some embodiments, the viral vector is an adeno-associated virus (AAV) vector. In some embodiments, the AAV is a AAV6 or AAV9 serotype.

In some embodiments, the viral vector comprises a genome editing system. In some embodiments, the genome editing system comprises a Cas peptide or a nucleic acid molecule encoding a Cas peptide and at least one gRNA molecule comprising a targeting domain that is complementary to a target sequence of a PD-1 gene or a nucleic acid molecule encoding at least one gRNA. In some embodiments, the Cas peptide is a Cas9 peptide. In some embodiments, the Cas9 peptide is a Staphylococcus aureus Cas9 (SaCas9) peptide. In some embodiments, the SaCas9 peptide is an adeno-associated virus (AAV)-compatible SaCas9.

In some embodiments, the at least one gRNA molecule comprises a nucleic acid sequence having at least about 50% identity, at least about 60% identity, at least about 70% identity, at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, or at least about 99% identity to a nucleotide sequence selected from the group consisting of SEQ ID NOs:1-5. In some embodiments, the gRNA molecules comprises a nucleotide sequence selected from the group consisting of SEQ ID NOs:1-5.

In some embodiments, the genome editing system comprises a Cas peptide or a nucleic acid molecule encoding a Cas peptide and at least two gRNA molecules comprising at least two targeting domains that are complementary to at least two target sequences of a PD-1 gene. In some embodiments, the at least two gRNA molecules comprise at least two sequences having at least about 50% identity, at least about 60% identity, at least about 70% identity, at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, or at least about 99% identity to at least two nucleotide sequences selected from the group consisting of SEQ ID NOs:1-5. In some embodiments, the at least two gRNA molecules comprises at least two nucleotide sequences selected from the group consisting of SEQ ID NOs:1-5.

In some embodiments, the viral vector comprises a plasmid. In some embodiments, the plasmid comprises a genome editing system. In some embodiments, the plasmid comprises a nucleic acid sequence encoding a Cas peptide and at least one nucleic acid sequence encoding at least one gRNA molecule comprising a targeting domain that is complementary to at least one target sequence of a PD-1 gene. In some embodiments, the Cas peptide is a Cas9 peptide. In some embodiments, the Cas9 peptide is a SaCas9 peptide. In some embodiments, the SaCas9 peptide is an AAV-compatible SaCas9. In some embodiments, the at least one gRNA molecule comprises a nucleic acid sequence having at least about 50% identity, at least about 60% identity, at least about 70% identity, at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, or at least about 99% identity to a nucleotide sequence selected from the group consisting of SEQ ID NOs:1-5. In some embodiments, the gRNA molecules comprises a nucleotide sequence selected from the group consisting of SEQ ID NOs:1-5.

In some embodiments, the plasmid comprises a nucleic acid sequence encoding a Cas peptide and at least two nucleic acid sequences encoding at least two gRNA molecules comprising at least two targeting domains that are complementary to at least two target sequences of a PD-1 gene. In some embodiments, the Cas peptide is a Cas9 peptide. In some embodiments, the Cas9 peptide is a SaCas9 peptide. In some embodiments, the SaCas9 peptide is an AAV-compatible SaCas9. In some embodiments, the at least two gRNA molecules comprise at least two sequences having at least about 50% identity, at least about 60% identity, at least about 70% identity, at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, or at least about 99% identity to at least two nucleotide sequences selected from the group consisting of SEQ ID NOs:1-5. In some embodiments, the at least two gRNA molecules comprises at least two nucleotide sequences selected from the group consisting of SEQ ID NOs:1-5.

In some embodiments, the invention relates to a method of reactivating exhausted T cells in a subject. In some embodiments, the T cells are CD8⁺ T cells. In some embodiments, the T cells are cytotoxic T lymphocytes (CTLs).

In some embodiments, the method comprises administering to the subject a genome editing system. In some embodiments, the genome editing system comprises a Cas peptide or a nucleic acid molecule encoding a Cas peptide and at least one gRNA molecule comprising a targeting domain that is complementary to a target sequence of a PD-1 gene or a nucleic acid molecule encoding at least one gRNA. In some embodiments, the Cas peptide is a Cas9 peptide. In some embodiments, the Cas9 peptide is a Staphylococcus aureus Cas9 (SaCas9) peptide. In some embodiments, the SaCas9 peptide is an adeno-associated virus (AAV)-compatible SaCas9.

In some embodiments, the at least one gRNA molecule comprises a nucleic acid sequence having at least about 50% identity, at least about 60% identity, at least about 70% identity, at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, or at least about 99% identity to a nucleotide sequence selected from the group consisting of SEQ ID NOs:1-5. In some embodiments, the gRNA molecules comprises a nucleotide sequence selected from the group consisting of SEQ ID NOs:1-5.

In some embodiments, the genome editing system comprises a Cas peptide or a nucleic acid molecule encoding a Cas peptide and at least two gRNA molecules comprising at least two targeting domains that are complementary to at least two target sequences of a PD-1 gene, at least one nucleic acid molecule encoding at least two gRNA molecules, or at least two nucleic acid molecules encoding at least two gRNA molecules. In some embodiments, the at least two gRNA molecules comprise at least two sequences having at least about 50% identity, at least about 60% identity, at least about 70% identity, at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, or at least about 99% identity to at least two nucleotide sequences selected from the group consisting of SEQ ID NOs:1-5. In some embodiments, the at least two gRNA molecules comprises at least two nucleotide sequences selected from the group consisting of SEQ ID NOs:1-5.

In some embodiments, the method comprises administering to the subject a plasmid. In some embodiments, the plasmid encodes a genome editing system. In some embodiments, the plasmid comprises a nucleic acid sequence encoding a Cas peptide and at least one nucleic acid sequence encoding at least one gRNA molecule comprising a targeting domain that is complementary to a target sequence of a PD-1 gene. In some embodiments, the Cas peptide is a Cas9 peptide. In some embodiments, the Cas9 peptide is a SaCas9 peptide. In some embodiments, the SaCas9 peptide is an AAV-compatible SaCas9. In some embodiments, the at least one gRNA molecule comprises a nucleic acid sequence having at least about 50% identity, at least about 60% identity, at least about 70% identity, at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, or at least about 99% identity to a nucleotide sequence selected from the group consisting of SEQ ID NOs:1-5. In some embodiments, the gRNA molecules comprises a nucleotide sequence selected from the group consisting of SEQ ID NOs:1-5.

In some embodiments, the plasmid comprises a nucleic acid sequence encoding a Cas peptide and at least two nucleic acid sequences encoding at least two gRNA molecules comprising at least two targeting domains that are complementary to at least two target sequences of a PD-1 gene. In some embodiments, the Cas peptide is a Cas9 peptide. In some embodiments, the Cas9 peptide is a SaCas9 peptide. In some embodiments, the SaCas9 peptide is an AAV-compatible SaCas9. In some embodiments, the at least two gRNA molecules comprise at least two sequences having at least about 50% identity, at least about 60% identity, at least about 70% identity, at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, or at least about 99% identity to at least two nucleotide sequences selected from the group consisting of SEQ ID NOs:1-5. In some embodiments, the at least two gRNA molecules comprises at least two nucleotide sequences selected from the group consisting of SEQ ID NOs:1-5.

In some embodiments, the method comprises administering to the subject a viral vector. In some embodiments, the viral vector is an adeno-associated virus (AAV) vector. In some embodiments, the AAV is a AAV6 or AAV9 serotype.

In some embodiments, the viral vector comprises a genome editing system. In some embodiments, the genome editing system comprises a Cas peptide or a nucleic acid molecule encoding a Cas peptide and at least one gRNA molecule comprising a targeting domain that is complementary to a target sequence of a PD-1 gene or a nucleic acid molecule encoding at least one gRNA. In some embodiments, the Cas peptide is a Cas9 peptide. In some embodiments, the Cas9 peptide is a Staphylococcus aureus Cas9 (SaCas9) peptide. In some embodiments, the SaCas9 peptide is an adeno-associated virus (AAV)-compatible SaCas9.

In some embodiments, the at least one gRNA molecule comprises a nucleic acid sequence having at least about 50% identity, at least about 60% identity, at least about 70% identity, at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, or at least about 99% identity to a nucleotide sequence selected from the group consisting of SEQ ID NOs:1-5. In some embodiments, the gRNA molecules comprises a nucleotide sequence selected from the group consisting of SEQ ID NOs:1-5.

In some embodiments, the genome editing system comprises a Cas peptide or a nucleic acid molecule encoding a Cas peptide and at least two gRNA molecules comprising at least two targeting domains that are complementary to at least two target sequences of a PD-1 gene. In some embodiments, the at least two gRNA molecules comprise at least two sequences having at least about 50% identity, at least about 60% identity, at least about 70% identity, at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, or at least about 99% identity to at least two nucleotide sequences selected from the group consisting of SEQ ID NOs:1-5. In some embodiments, the at least two gRNA molecules comprises at least two nucleotide sequences selected from the group consisting of SEQ ID NOs:1-5.

In some embodiments, the viral vector comprises a plasmid. In some embodiments, the plasmid comprises a genome editing system. In some embodiments, the plasmid comprises a nucleic acid sequence encoding a Cas peptide and at least one nucleic acid sequence encoding at least one gRNA molecule comprising a targeting domain that is complementary to at least one target sequence of a PD-1 gene. In some embodiments, the Cas peptide is a Cas9 peptide. In some embodiments, the Cas9 peptide is a SaCas9 peptide. In some embodiments, the SaCas9 peptide is an AAV-compatible SaCas9. In some embodiments, the at least one gRNA molecule comprises a nucleic acid sequence having at least about 50% identity, at least about 60% identity, at least about 70% identity, at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, or at least about 99% identity to a nucleotide sequence selected from the group consisting of SEQ ID NOs:1-5. In some embodiments, the gRNA molecules comprises a nucleotide sequence selected from the group consisting of SEQ ID NOs:1-5.

In some embodiments, the plasmid comprises a nucleic acid sequence encoding a Cas peptide and at least two nucleic acid sequences encoding at least two gRNA molecules comprising at least two targeting domains that are complementary to at least two target sequences of a PD-1 gene. In some embodiments, the Cas peptide is a Cas9 peptide. In some embodiments, the Cas9 peptide is a SaCas9 peptide. In some embodiments, the SaCas9 peptide is an AAV-compatible SaCas9. In some embodiments, the at least two gRNA molecules comprise at least two sequences having at least about 50% identity, at least about 60% identity, at least about 70% identity, at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, or at least about 99% identity to at least two nucleotide sequences selected from the group consisting of SEQ ID NOs:1-5. In some embodiments, the at least two gRNA molecules comprises at least two nucleotide sequences selected from the group consisting of SEQ ID NOs:1-5.

In some embodiments, the invention relates to a method of treating or preventing a disease or disorder in a subject. In some embodiments, the disease or disorder is an infection, a condition associated with an infection, and cancer. In some embodiments, the infection is a chronic infection. In some embodiments, the chronic infection is associated with immune cell exhaustion. In some embodiments, the infection is an infection with a viral pathogen. In some embodiments, the virus is selected from the group consisting of human immunodeficiency virus 1 (HIV-1), hepatitis B virus (HBV), hepatitis C virus (HCV), human T-lymphotropic virus 1 (HTLV-1), and influenza. In some embodiments, the infection is an infection with a non-viral pathogen. In some embodiments, the non-viral pathogen is selected from the group consisting of Mycobacterium tuberculosis, a Plasmodium species, a Listeria species, a Toxoplasma species, and a Leishmania species.

In some embodiments, a condition associated with an infection is a condition associated with a chronic infection or a condition associated with a chronic infection associated with immune cell exhaustion. In some embodiments, the condition is acquired immunodeficiency syndrome (AIDS).

In some embodiments, the method comprises administering to the subject a genome editing system. In some embodiments, the genome editing system comprises a Cas peptide or a nucleic acid molecule encoding a Cas peptide and at least one gRNA molecule comprising a targeting domain that is complementary to a target sequence of a PD-1 gene or a nucleic acid molecule encoding at least one gRNA. In some embodiments, the Cas peptide is a Cas9 peptide. In some embodiments, the Cas9 peptide is a Staphylococcus aureus Cas9 (SaCas9) peptide. In some embodiments, the SaCas9 peptide is an adeno-associated virus (AAV)-compatible SaCas9.

In some embodiments, the at least one gRNA molecule comprises a nucleic acid sequence having at least about 50% identity, at least about 60% identity, at least about 70% identity, at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, or at least about 99% identity to a nucleotide sequence selected from the group consisting of SEQ ID NOs:1-5. In some embodiments, the gRNA molecules comprises a nucleotide sequence selected from the group consisting of SEQ ID NOs:1-5.

In some embodiments, the genome editing system comprises a Cas peptide or a nucleic acid molecule encoding a Cas peptide and at least two gRNA molecules comprising at least two targeting domains that are complementary to at least two target sequences of a PD-1 gene, at least one nucleic acid molecule encoding at least two gRNA molecules, or at least two nucleic acid molecules encoding at least two gRNA molecules. In some embodiments, the at least two gRNA molecules comprise at least two sequences having at least about 50% identity, at least about 60% identity, at least about 70% identity, at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, or at least about 99% identity to at least two nucleotide sequences selected from the group consisting of SEQ ID NOs:1-5. In some embodiments, the at least two gRNA molecules comprises at least two nucleotide sequences selected from the group consisting of SEQ ID NOs:1-5.

In some embodiments, the method comprises administering to the subject a plasmid. In some embodiments, the plasmid encodes a genome editing system. In some embodiments, the plasmid comprises a nucleic acid sequence encoding a Cas peptide and at least one nucleic acid sequence encoding at least one gRNA molecule comprising a targeting domain that is complementary to a target sequence of a PD-1 gene. In some embodiments, the Cas peptide is a Cas9 peptide. In some embodiments, the Cas9 peptide is a SaCas9 peptide. In some embodiments, the SaCas9 peptide is an AAV-compatible SaCas9. In some embodiments, the at least one gRNA molecule comprises a nucleic acid sequence having at least about 50% identity, at least about 60% identity, at least about 70% identity, at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, or at least about 99% identity to a nucleotide sequence selected from the group consisting of SEQ ID NOs:1-5. In some embodiments, the gRNA molecules comprises a nucleotide sequence selected from the group consisting of SEQ ID NOs:1-5.

In some embodiments, the plasmid comprises a nucleic acid sequence encoding a Cas peptide and at least two nucleic acid sequences encoding at least two gRNA molecules comprising at least two targeting domains that are complementary to at least two target sequences of a PD-1 gene. In some embodiments, the Cas peptide is a Cas9 peptide. In some embodiments, the Cas9 peptide is a SaCas9 peptide. In some embodiments, the SaCas9 peptide is an AAV-compatible SaCas9. In some embodiments, the at least two gRNA molecules comprise at least two sequences having at least about 50% identity, at least about 60% identity, at least about 70% identity, at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, or at least about 99% identity to at least two nucleotide sequences selected from the group consisting of SEQ ID NOs:1-5. In some embodiments, the at least two gRNA molecules comprises at least two nucleotide sequences selected from the group consisting of SEQ ID NOs:1-5.

In some embodiments, the method comprises administering to the subject a viral vector. In some embodiments, the viral vector is an adeno-associated virus (AAV) vector. In some embodiments, the AAV is a AAV6 or AAV9 serotype.

In some embodiments, the viral vector comprises a genome editing system. In some embodiments, the genome editing system comprises a Cas peptide or a nucleic acid molecule encoding a Cas peptide and at least one gRNA molecule comprising a targeting domain that is complementary to a target sequence of a PD-1 gene or a nucleic acid molecule encoding at least one gRNA. In some embodiments, the Cas peptide is a Cas9 peptide. In some embodiments, the Cas9 peptide is a Staphylococcus aureus Cas9 (SaCas9) peptide. In some embodiments, the SaCas9 peptide is an adeno-associated virus (AAV)-compatible SaCas9.

In some embodiments, the at least one gRNA molecule comprises a nucleic acid sequence having at least about 50% identity, at least about 60% identity, at least about 70% identity, at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, or at least about 99% identity to a nucleotide sequence selected from the group consisting of SEQ ID NOs:1-5. In some embodiments, the gRNA molecules comprises a nucleotide sequence selected from the group consisting of SEQ ID NOs:1-5.

In some embodiments, the genome editing system comprises a Cas peptide or a nucleic acid molecule encoding a Cas peptide and at least two gRNA molecules comprising at least two targeting domains that are complementary to at least two target sequences of a PD-1 gene. In some embodiments, the at least two gRNA molecules comprise at least two sequences having at least about 50% identity, at least about 60% identity, at least about 70% identity, at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, or at least about 99% identity to at least two nucleotide sequences selected from the group consisting of SEQ ID NOs:1-5. In some embodiments, the at least two gRNA molecules comprises at least two nucleotide sequences selected from the group consisting of SEQ ID NOs:1-5.

In some embodiments, the viral vector comprises a plasmid. In some embodiments, the plasmid comprises a genome editing system. In some embodiments, the plasmid comprises a nucleic acid sequence encoding a Cas peptide and at least one nucleic acid sequence encoding at least one gRNA molecule comprising a targeting domain that is complementary to at least one target sequence of a PD-1 gene. In some embodiments, the Cas peptide is a Cas9 peptide. In some embodiments, the Cas9 peptide is a SaCas9 peptide. In some embodiments, the SaCas9 peptide is an AAV-compatible SaCas9. In some embodiments, the at least one gRNA molecule comprises a nucleic acid sequence having at least about 50% identity, at least about 60% identity, at least about 70% identity, at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, or at least about 99% identity to a nucleotide sequence selected from the group consisting of SEQ ID NOs:1-5. In some embodiments, the gRNA molecules comprises a nucleotide sequence selected from the group consisting of SEQ ID NOs:1-5.

In some embodiments, the plasmid comprises a nucleic acid sequence encoding a Cas peptide and at least two nucleic acid sequences encoding at least two gRNA molecules comprising at least two targeting domains that are complementary to at least two target sequences of a PD-1 gene. In some embodiments, the Cas peptide is a Cas9 peptide. In some embodiments, the Cas9 peptide is a SaCas9 peptide. In some embodiments, the SaCas9 peptide is an AAV-compatible SaCas9. In some embodiments, the at least two gRNA molecules comprise at least two sequences having at least about 50% identity, at least about 60% identity, at least about 70% identity, at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, or at least about 99% identity to at least two nucleotide sequences selected from the group consisting of SEQ ID NOs:1-5. In some embodiments, the at least two gRNA molecules comprises at least two nucleotide sequences selected from the group consisting of SEQ ID NOs:1-5.

In some embodiments, the method further comprises administering to the subject at least one additional therapeutic. In some embodiments, the at least one additional therapeutic is selected from the group consisting of antivirals, antibiotics, antifungals, antiparasitics, and anti-cancer agents. Examples of suitable antivirals include, but are not limited to, nucleoside reverse transcriptase inhibitors (NRTIs, e.g., abacavir, emtricitabine, lamuvidine, tenofovir disoproxil fumarate, zidovudine, entecavir, etc.), non-nucleoside reverse transcriptase inhibitors (NNRTIs, e.g., doravirine, efavirenz, etravirine, nevirapine, rilpivirine, adefovir, etc.), protease inhibitors (PIs, e.g., atazanavir, darunavir, fosamprenavir, ritonavir, tipranavir, grazoprevir, glecaprevir, voxilaprevir, etc.), fusion inhibitors (e.g., enfuvirtide, etc.), CCR5 antagonists (e.g., maraviroc, etc.), integrase strand transfer inhibitors (NSTIs, e.g., cabotegravir, dolutegravir, raltegravir, etc.), attachment inhibitors (e.g., fostemsavir, etc.), post-attachment inhibitors (e.g., ibalizumab-uiyk, etc.), capsid inhibitors (e.g., lenacapavir, etc.), pharmacokinetic enhancers (e.g., cobicistat, etc.), nucleoside analogs (e.g., ribavirin, sofosbuvir, telbivudine, etc.), neuraminidase inhibitors (e.g., oseltamivir, zanamivir, peramivir, etc.), protein inhibitors (e.g., elbasvir, pibrentasvir, ledipasvir, velpatasvir, baloxavir marboxil, etc.). Examples of suitable antibiotics include, but are not limited to, isoniazid, rifampin, pyrazinamide, ethambutol, bedaquiline, linezolid, pretomanid, rifapentine, cycloserine, ampicillin, gentamicin, trimethoprim-sulfamethoxazole, linezolid, levofloxacin, penicillin, meropenem, vancomycin, and azithromycin. Examples of suitable antiparasitics include, but are not limited to, chloroquine, hydroxychloroquine, artemisinin, quinine, primaquine, artesunate, artemether, lumefantrine, atovaquone, proguanil, mefloquine, tetracycline, clindamycin, pyrimethamine, sulfadiazine, atovaquone, spiramycin, leucovorin, miltefosine, amphotericin B, sodium stibogluconate, paromomycin, pentamidine isethionate, ketoconazole, fluconazole, and meglumine antimonate. Examples of suitable cancer therapies include, but are not limited to, CAR-T cells and CAR-NK cells.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1: CRISPR-Mediated Knockout of PD-1 Gene in HIV-1-Specific Cytotoxic T Cells

CRISPR-Cas9 technology allows specific and efficient editing of human genes and is widely used to knockout endogenous TCR gene expression in CAR-T cells for cancer immunotherapy. Here, a CRISPR-Cas9-based strategy to knockout PD-1 gene expression in CTLs was developed and validated.

Design and Selection of CRISPR gRNAs

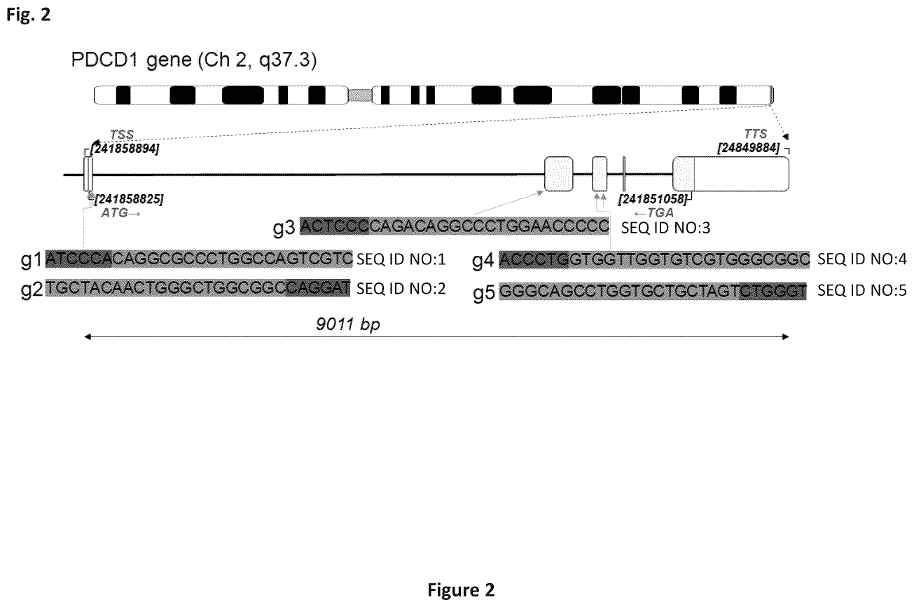

The human PDCD1 (PD-1) gene coding sequence was screened using the CRISPR design tool (CRISPOR) for the presence of gRNAs binding sites for AAV-compatible SaCas9 (NNGRRT PAM sequence). A set of five gRNAs targeting exons 1-3 of the PDCD1 gene having the highest ON-target efficiency and the lowest OFF-target scores were selected (FIG. 2, Table 1).

TABLE 1
List of CRISPR-SaCas9 gRNAs targeting
coding sequence of PDCD1 (PD-1) gene.

| | | | | Coordinates |
|---|---|---|---|---|
| | | Sequence | | in |
| Name | Strand | (5′-3′) | PAM | GRCh38.p14* |
| PD-1 g1 | -1 | GACGACTGGCCAGG | TGGGAT | 241858806- |
| (63rev) | | GCGCCTG | | 241858826 |
| | | (SEQ ID NO: 1) | | |
| PD-1 g2 | 1 | TGCTACAACTGGGC | CAGGAT | 241858778- |
| (118forw) | | TGGCGGC | | 241858798 |
| | | (SEQ ID NO: 2) | | |
| PD-1 g3 | -1 | GGGGGTTCCAGGGC | GGGAGT | 241852954- |
| (175rev) | | CTGTCTG | | 241852974 |
| | | (SEQ ID NO: 3) | | |
| PD-1 g4 | -1 | GCCGCCCACGACAC | CAGGGT | 241852262- |
| (657rev) | | CAACCAC | | 241852282 |
| | | (SEQ ID NO: 4) | | |
| PD-1 g5 | 1 | GGGCAGCCTGGTGC | CTGGGT | 241852236- |
| (710forw) | | TGCTAGT | | 241852256 |
| | | (SEQ ID NO: 5) | | |

*human reference genome, chromosome 2

Verification of CRISPR gRNAs Targeting PD-1 Gene in Jurkat T Lymphocytic Cell Line

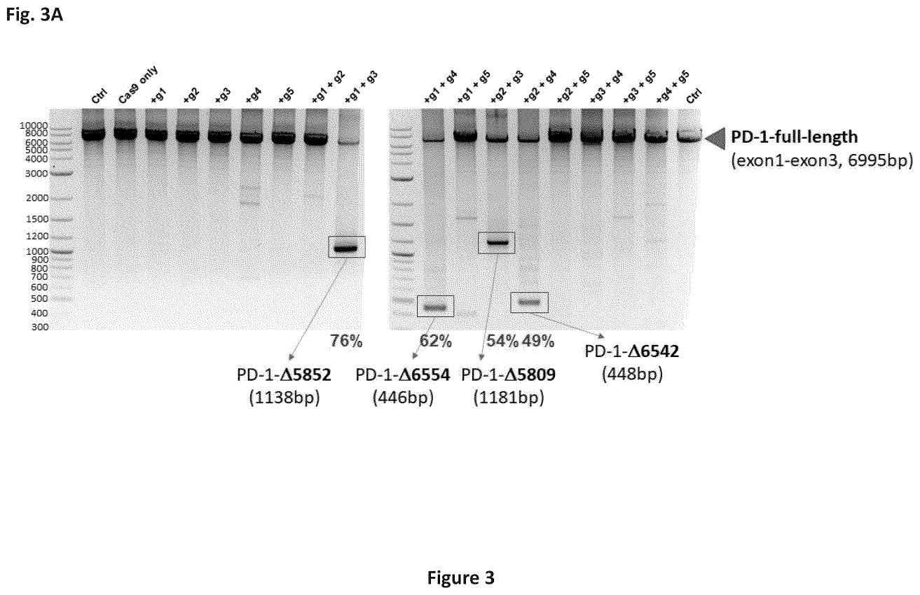

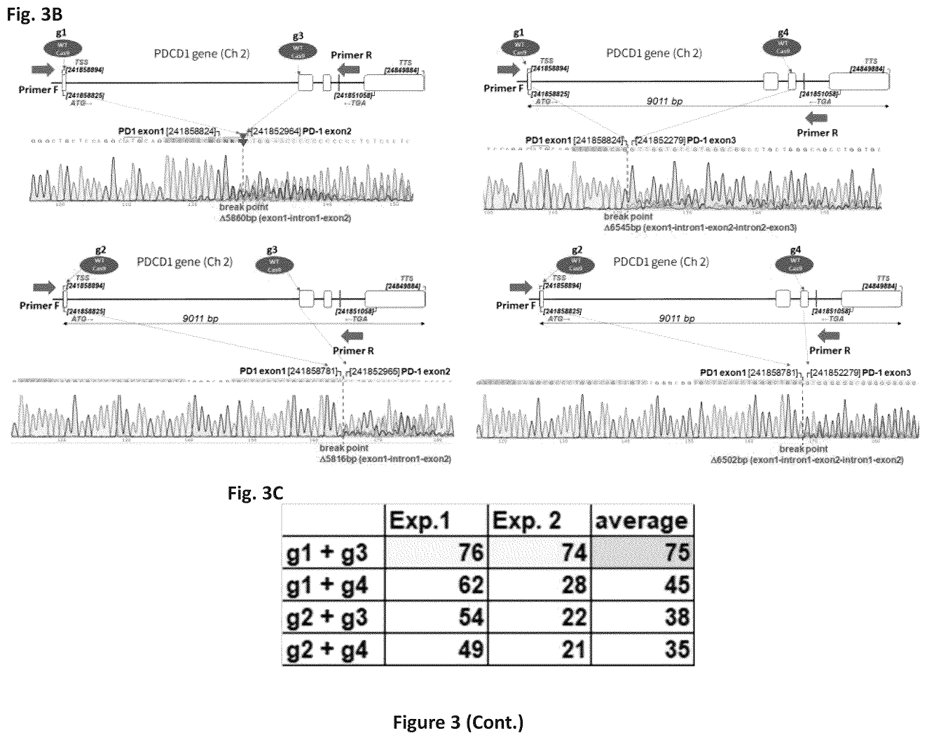

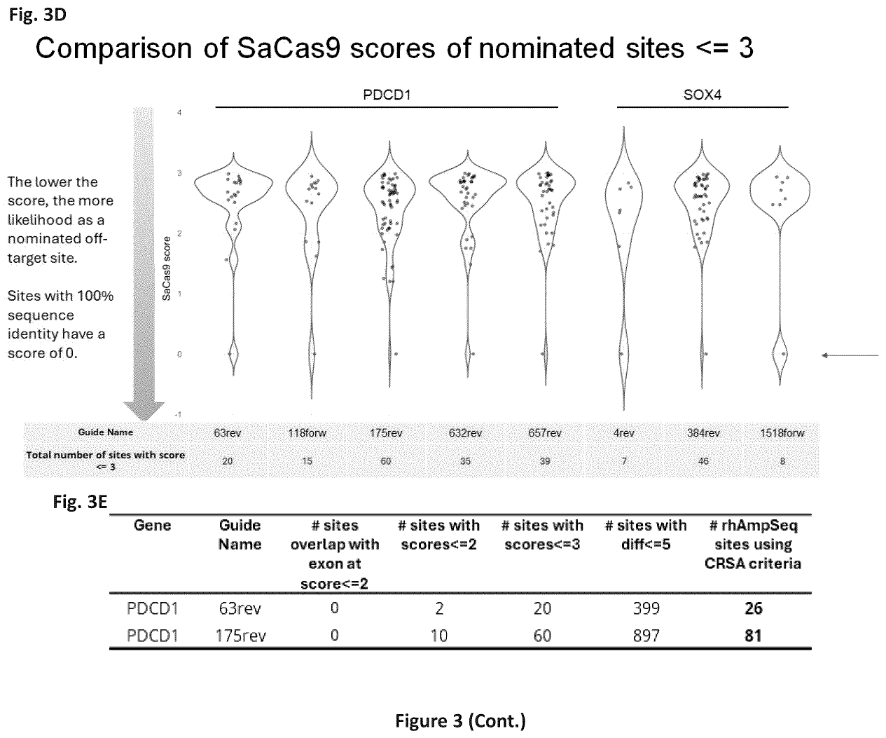

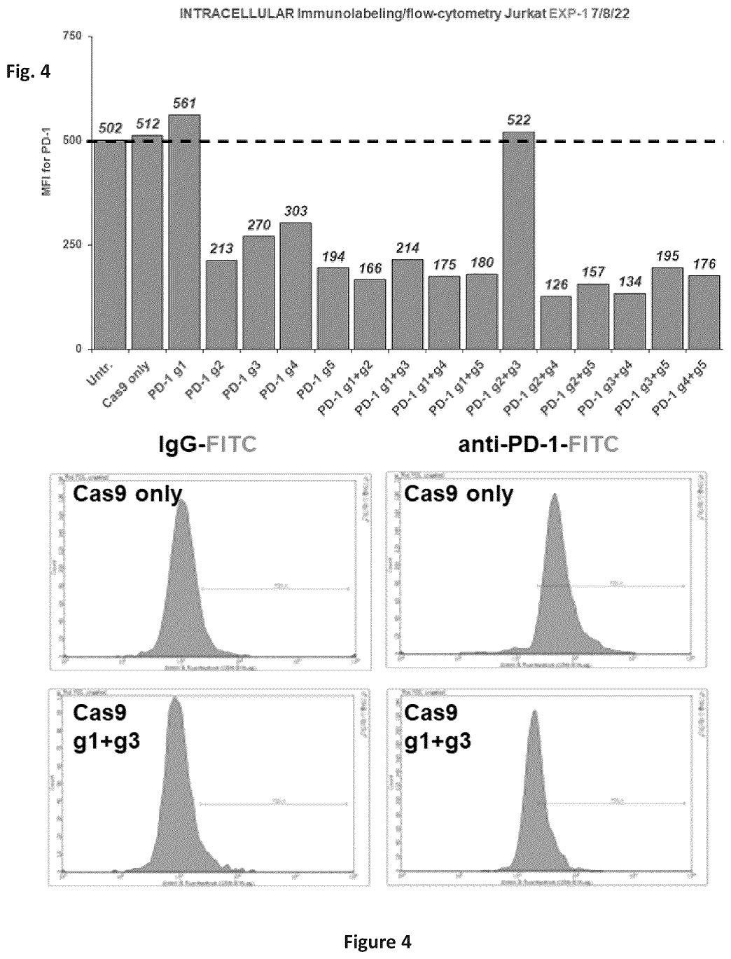

Next, candidate gRNAs were tested by electroporating ribonucleoprotein complexes composed of recombinant SaCas9 (Aldevron) and synthetic gRNAs (Synthego) into the Jurkat T-lymphoid cell line. Three days post-electroporation genomic DNA was prepared and analyzed by PCR using primers specific to the exon 1-exon 3 region of the PDCD1 gene (FIG. 3A). The CRISPR-mediated specific excision of the PDCD1 gene was confirmed by Sanger sequencing (FIG. 3B). The experiment was repeated and the pair of gRNAs g1+g3 was identified to give the most robust and consistent excision of the PDCD1 gene (FIG. 3C). In parallel, PD-1 expression levels were confirmed by immunolabeling with FITC-labeled anti-PD-1 antibodies followed by flow cytometry (FIG. 4).

Validation of the Best CRISPR gRNAs Pairs in Primary CD8⁺ T Cells

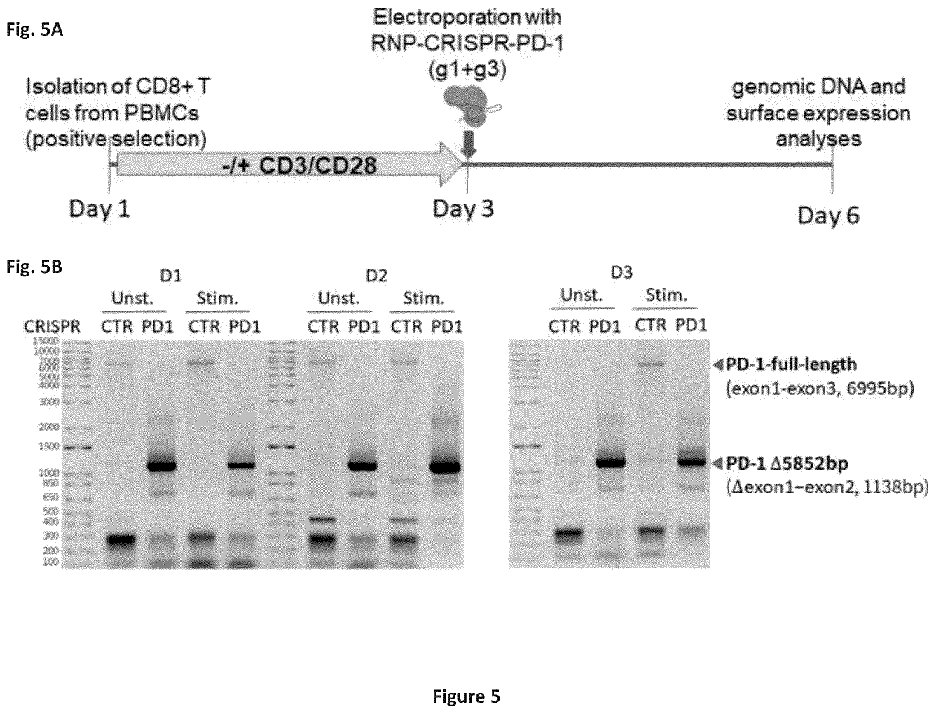

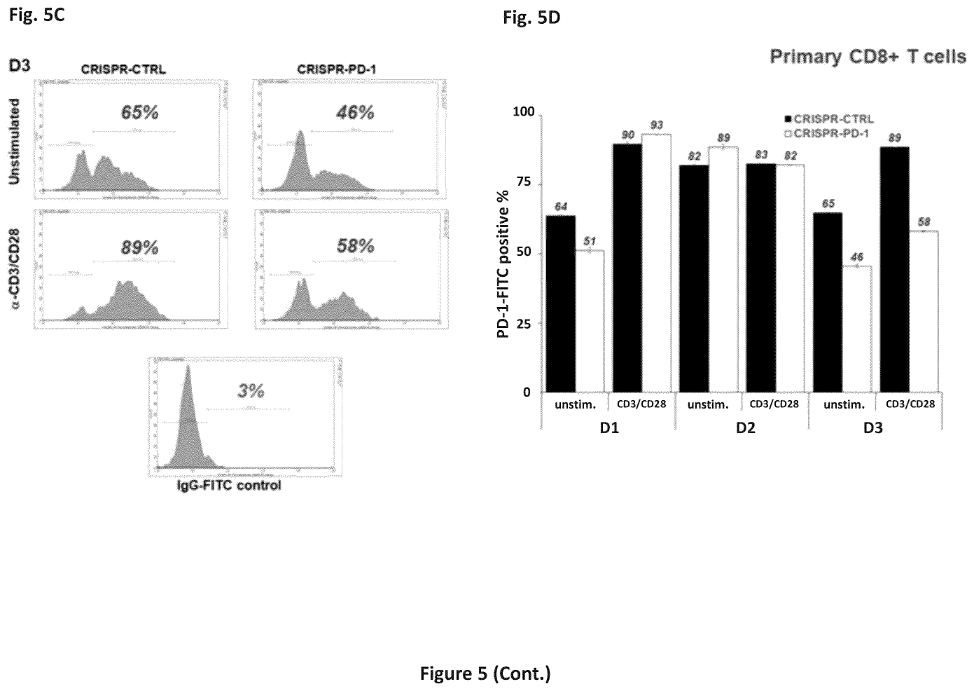

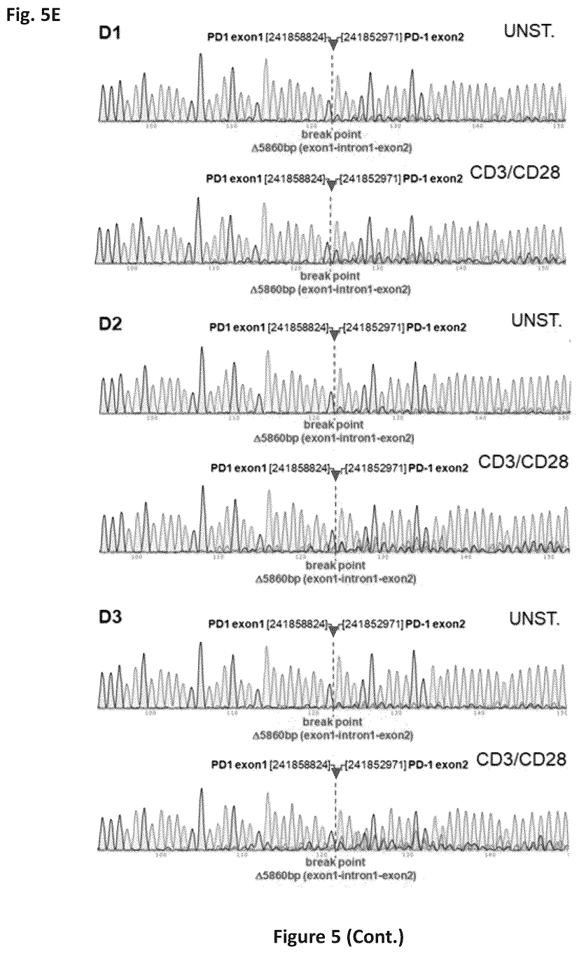

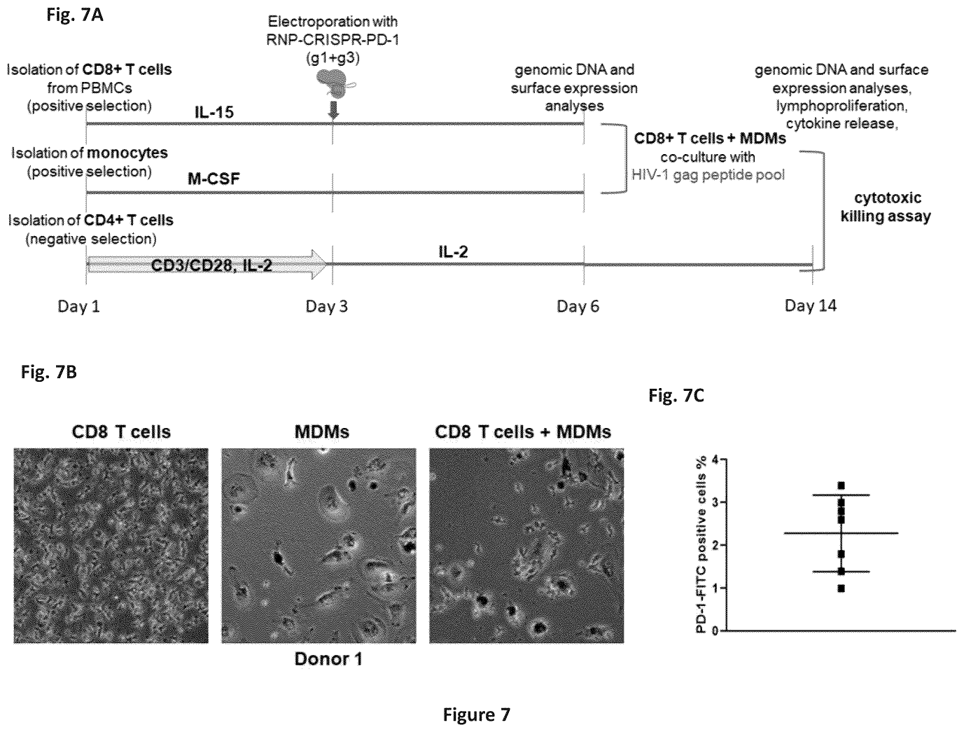

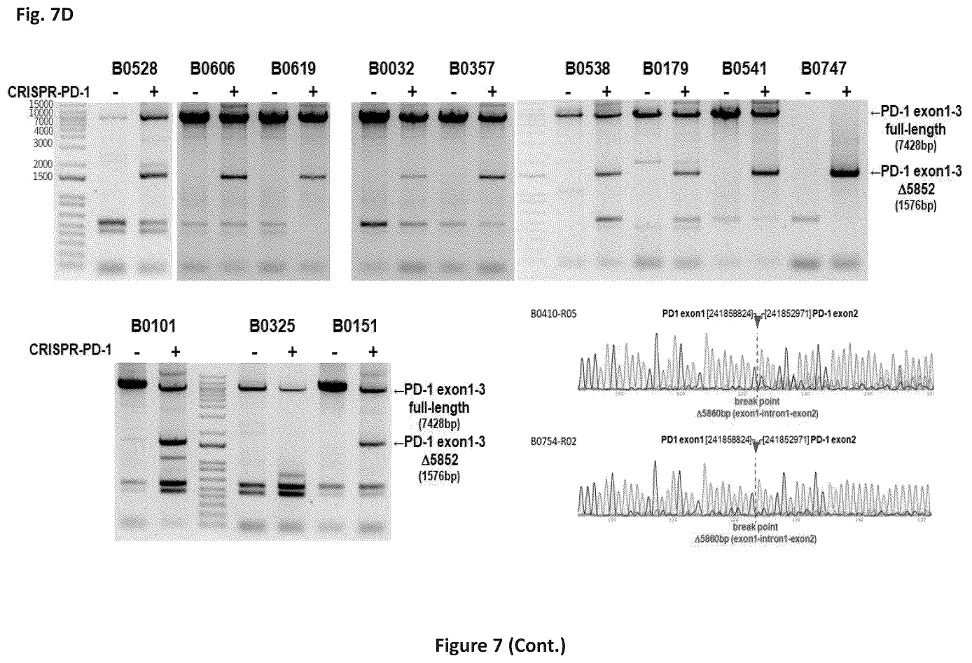

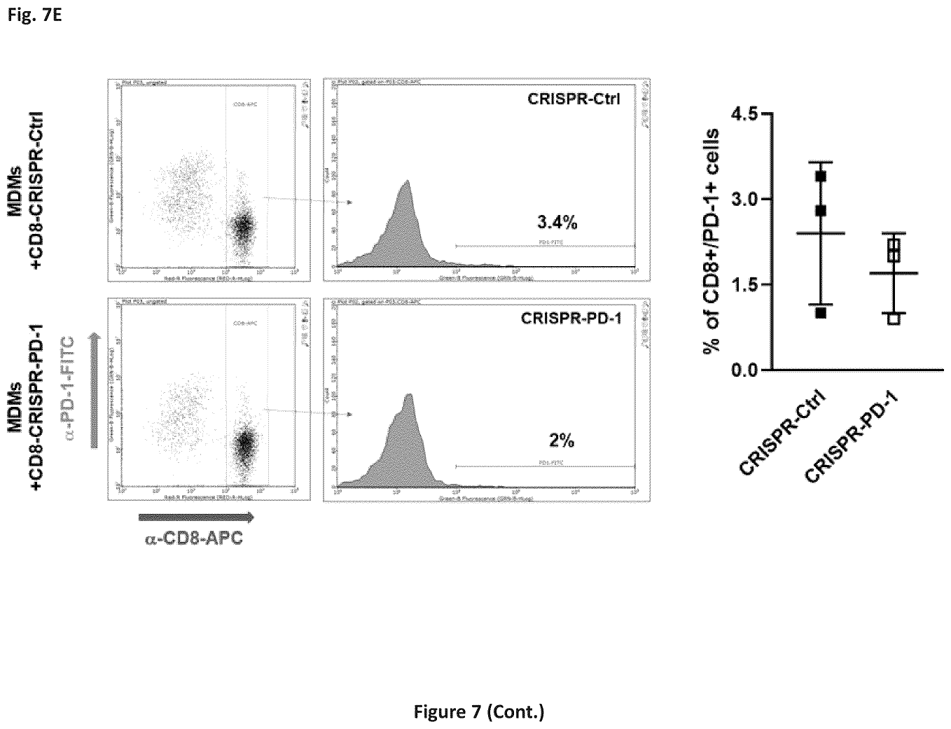

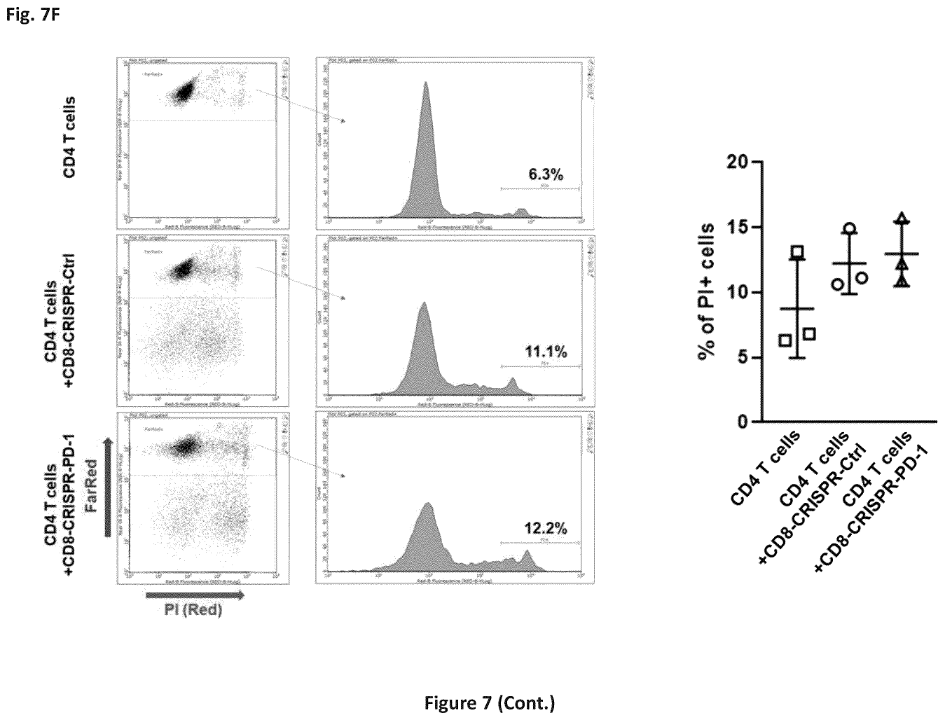

The most efficient gRNA combination (g1+g3) was validated in experiments using primary CD8⁺ T cells from three healthy donors. After isolation and three-day activation/expansion (FIG. 5A) cells were electroporated with ribonucleoprotein complexes composed of recombinant SaCas9 (Aldevron) and synthetic gRNAs g1+g3 (Synthego). CRISPR-mediated excision of the PDCD1 gene was evaluated by PCR genotyping and Sanger sequencing (FIG. 5B, C). PD-1 surface expression was checked by immunolabeling/flow cytometry (FIG. 5D). Excision of PD-1 gene in CD8⁺ T cells from people living with HIV-1. Peripheral blood mononuclear cells (PBMCs) from people with HIV on ART were obtained and used to isolate CD8⁺ T cells. CD8⁺ T cells were electroporated with RNP-CRISPR-PD-1 (g1+g3) and then co-cultured with isogenic monocyte-derived macrophages (MDMs) in the presence of HIV-1 Gag peptide pool to induce expansion of HIV-1 specific CTLs. Three days after electroporation, some cells were harvested for genomic DNA and CRISPR-mediated excision of the PDCD1 gene was evaluated by PCR genotyping and Sanger sequencing as before. The excision of the PDCD1 gene was verified for eleven out of twelve different HIV-positive donors (FIG. 7D).

Steps for Single gRNA Cloning into AAV-CRISPR Vector

Oligonucleotides representing protospacer regions+Bsa1 restriction enzyme overhangs of selected gRNAs PD-1 g1 and g3 were annealed, phosphorylated, and then ligated into Bsa1 digested backbone pX601 vector resulting in pX601-PD-1-g1 and pX601-PD-1-g3 single gRNA vectors. After transformation, single bacteria colonies were inoculated for minipreps. In parallel the same bacteria were used for direct PCRs using primers specific to the U6 promoter (forward) and bottom gRNA protospacer oligo (reverse). Next, plasmid DNA was isolated from the bacterial colonies positive for U6-gRNA amplicons and sent for Sanger sequencing to verify correctly cloned gRNAs protospacers.

Generating Dual gRNA CRISPR Vector

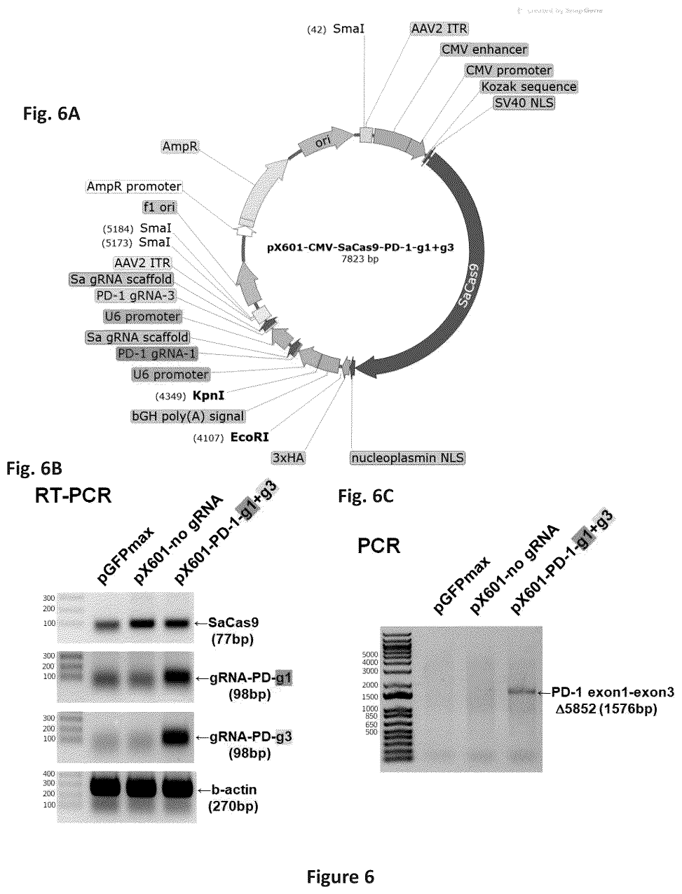

The U6-PD-1-g1-scaffold expression cassette was PCR amplified with primers carrying homology arms to EcoR1 and Kpn1 flanks of pX601 plasmid and inserted into EcoR1-Kpn1 digested pX601-PD-1-g3 vector using In-Fusion Snap Assembly kit resulting in dual gRNA pX601-PD-1-g1+g3 vector (FIG. 6A). After transformation single bacteria colonies were inoculated for minipreps and plasmid DNA from 10 colonies was isolated and sent for Sanger sequencing for verification. The final plasmid containing both gRNA g1 and g3 in correct orientation and order was then prepared via MaxiPrep to >1 mg for AAV packaging.

Verifying Functionality of Dual gRNA Plasmid

The myeloid cell line K562 cells were electroporated with pX601-no gRNA (control) or pX601-CMV-SaCas9-PD-1 g1+g3 plasmids and pGFPmax for transfection efficiency control. 48 h later cells were harvested, and RNA and genomic DNA were extracted. The expression of SaCas9 and gRNAs and CRISPR-mediated excision of the PD-1 gene were validated by RT-PCRs (FIG. 6B) and PCR (FIG. 6C). The most efficient SaCas9 gRNAs targeting the PDCD1 gene were identified and the “all-in-one” AAV-delivery vector carrying SaCas9 and pair of gRNAs (PD-1 g1+g3) was generated, tested, and packaged into AAV6 and AAV9 delivery vectors.

AAV6/AAV9-PD-1 g1+g3 vectors are tested in vivo in humanized mice model called MISTRG. Experiments using HIV-1-infected, ART-treated MISTRG mice are conducted.

### Developing an In Vitro Assay Using Primary Immune Cells

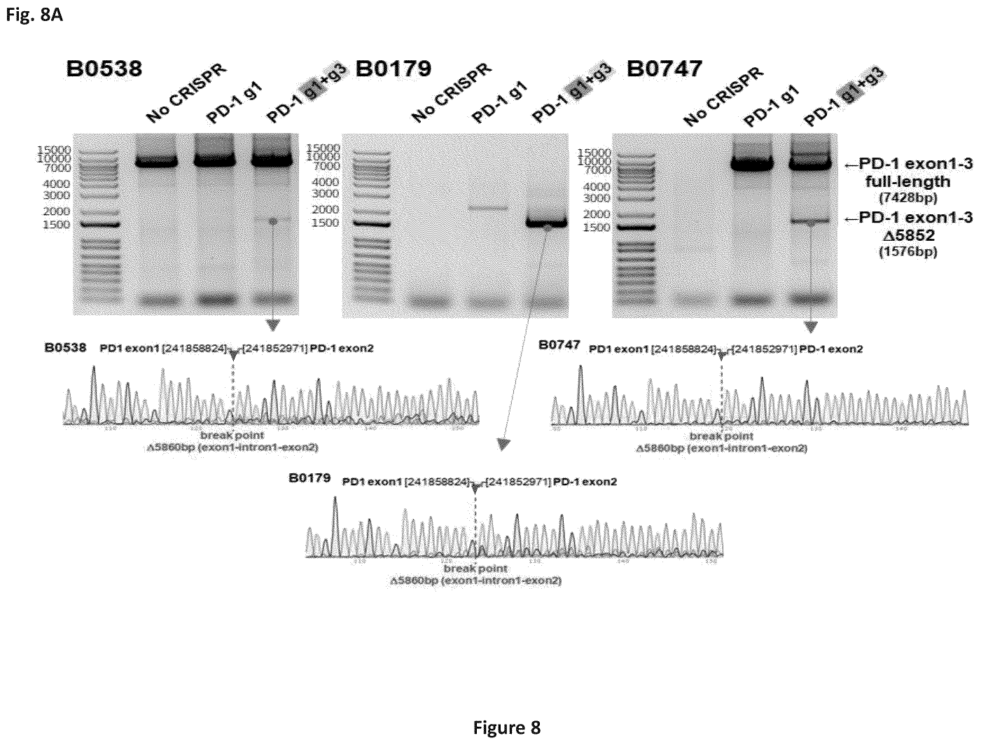

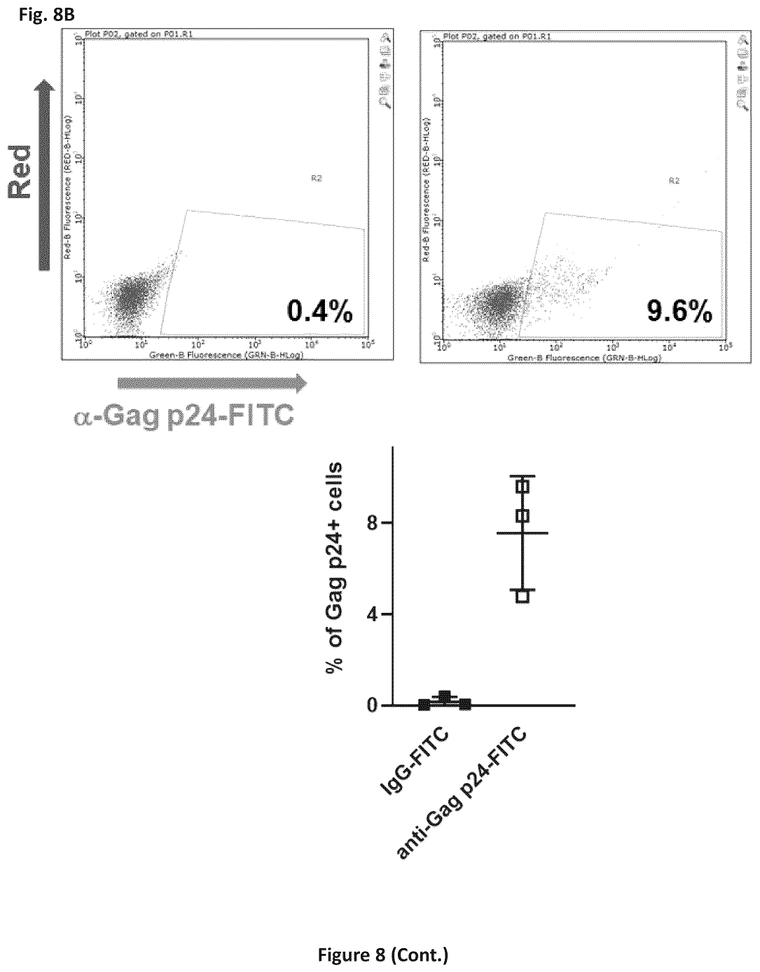

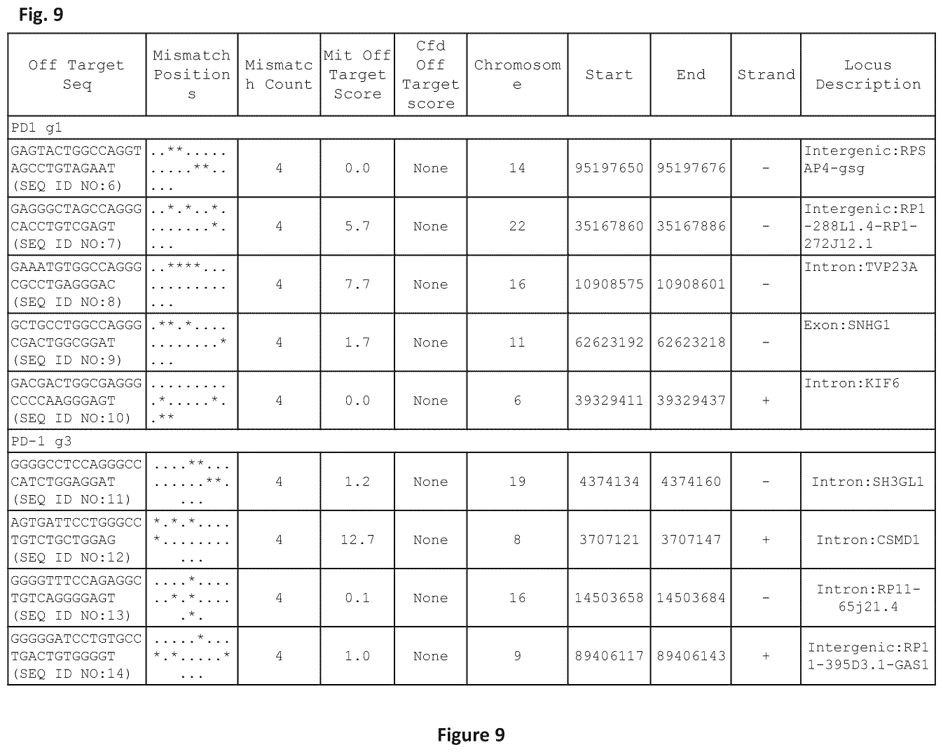

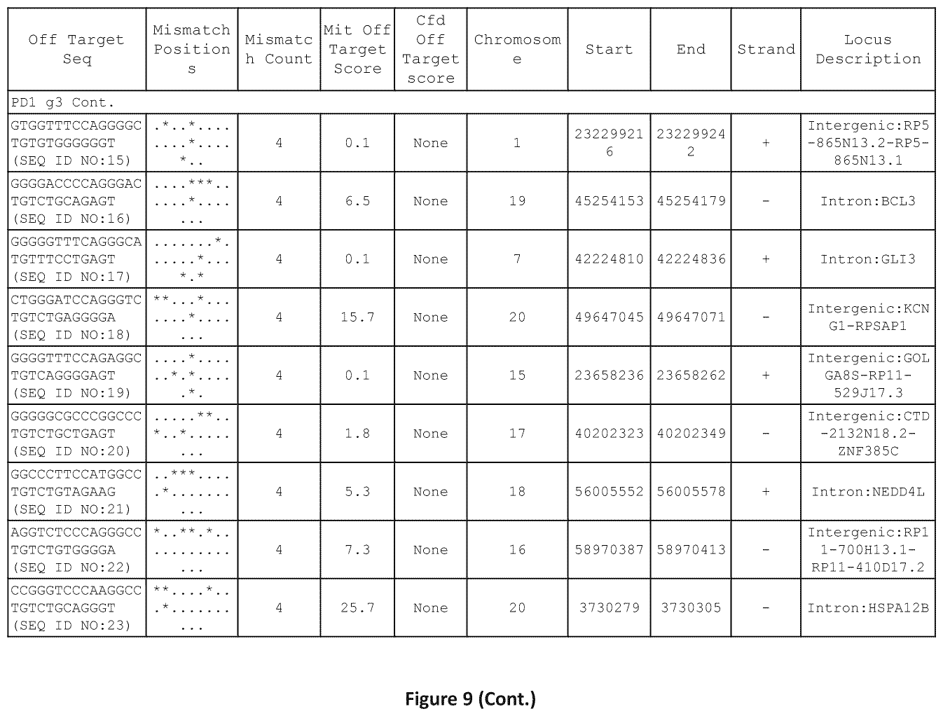

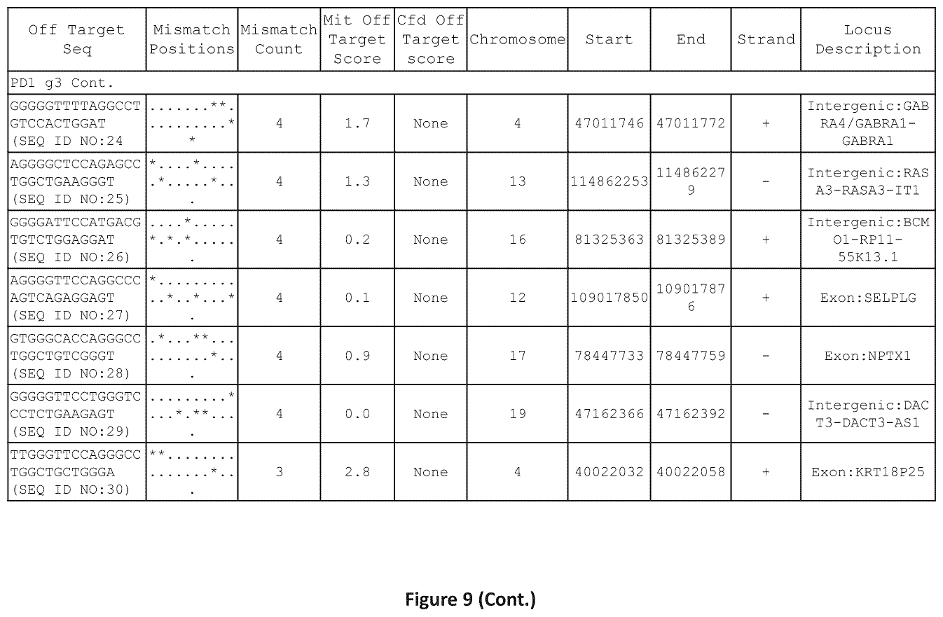

Peripheral blood mononuclear cells (PBMCs) from subjects with HIV on ART were obtained and CD8⁺ T cells, CD14⁺ monocytes, and CD4⁺ T cells isolated. CD8⁺ T cells were electroporated with RNP-CRISPR-PD-1 (g1+g3) and co-cultured with isogenic monocyte-derived macrophages (MDMs) in the presence of HIV-1 Gag peptide pool to induce expansion of HIV-1 specific CTLs. After one week, CD8⁺ T cells were collected and mixed in a 1:1 ratio with isogenic FarRed stained CD4⁺ T cells. After four hours, cells were collected and cytotoxic killing of CD4⁺ analyzed by propidium iodide staining followed by flow-cytometry (FIG. 7 and FIG. 8). Off-target analysis resulting from the guides also examined (FIG. 9).

### Validation in Healthy Donor Cells

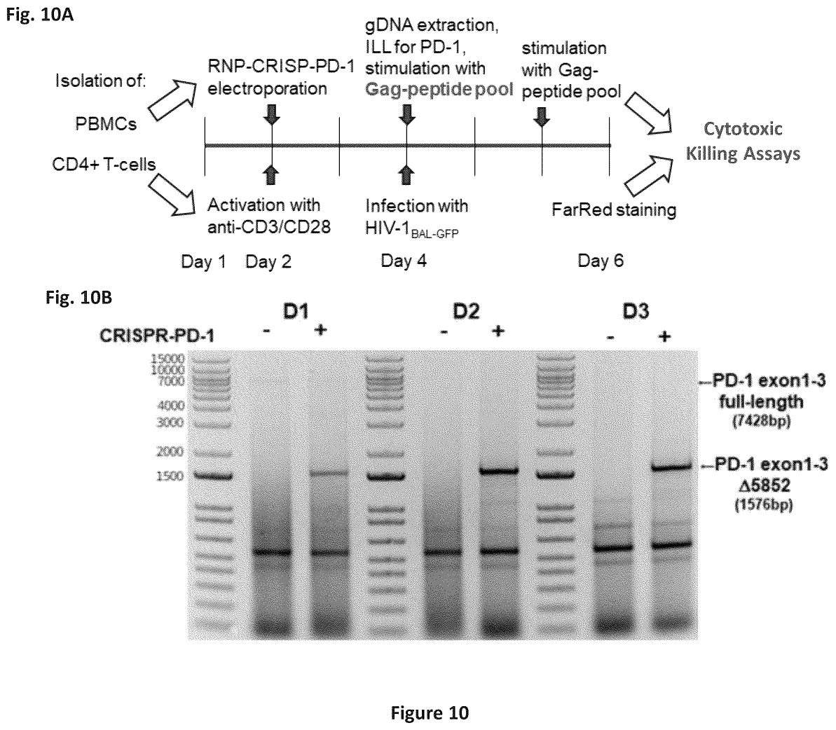

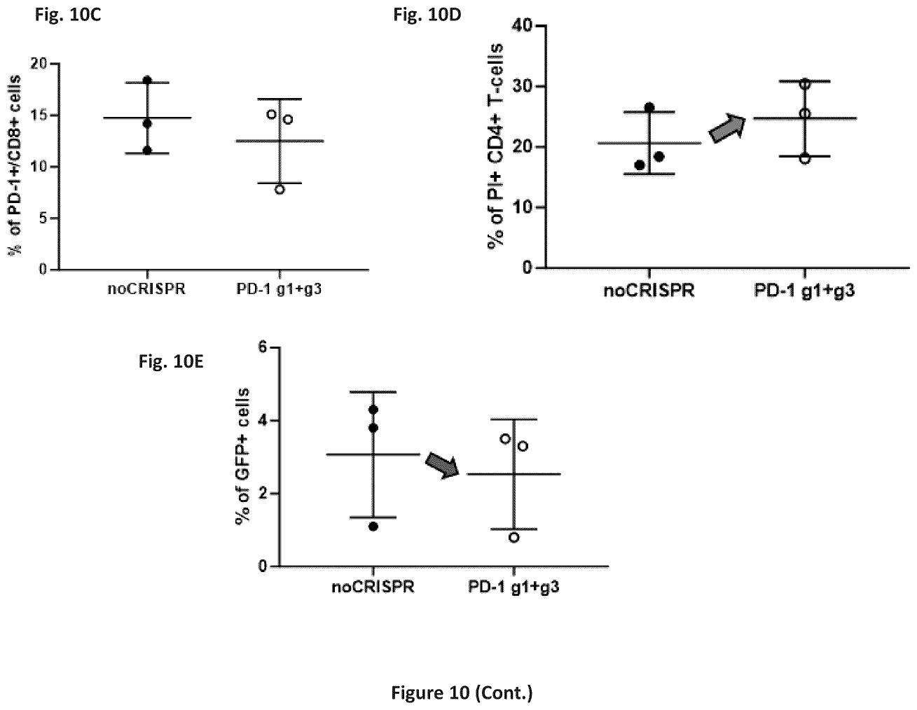

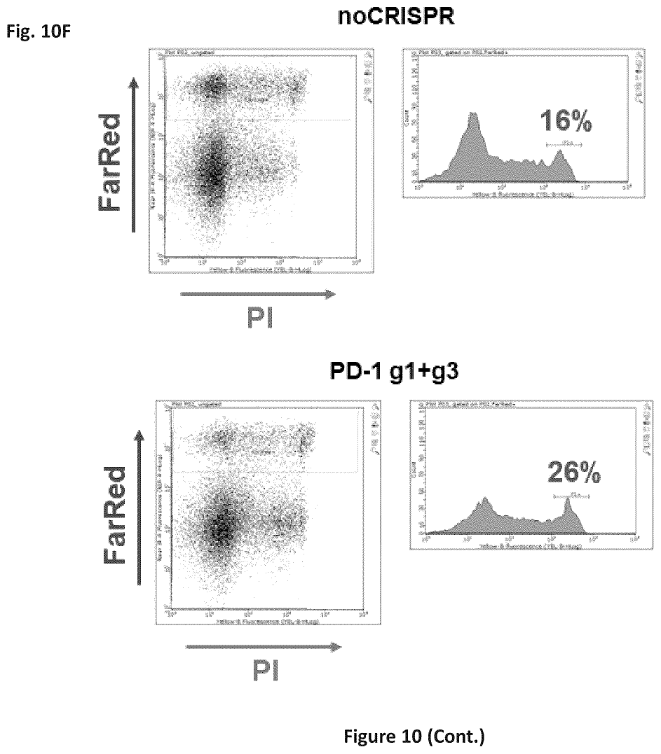

Peripheral blood mononuclear cells (PBMCs) from healthy subjects were obtained and CD4⁺ T cells were isolated. CD4⁺ T cells—depleted PBMCs (including CD8⁺ T cells and other remaining immune cell subtypes, were electroporated with RNP-CRISPR-PD-1 (g1+g3) and cultured in the presence of HIV-1 Gag peptide pool to induce expansion of HIV-1 specific CTLs. After three days, PBMCs were collected and mixed in a 1:1 ratio with isogenic FarRed stained CD4⁺ T cells that were infected with an HIV-1-GFP reporter virus. After four hours, cells were collected and cytotoxic killing of CD4⁺ analyzed by propidium iodide staining and GFP fluorescence followed by flow-cytometry (FIG. 10).

### In Vivo Validation of AAV-Delivered CRISPR-PD-1

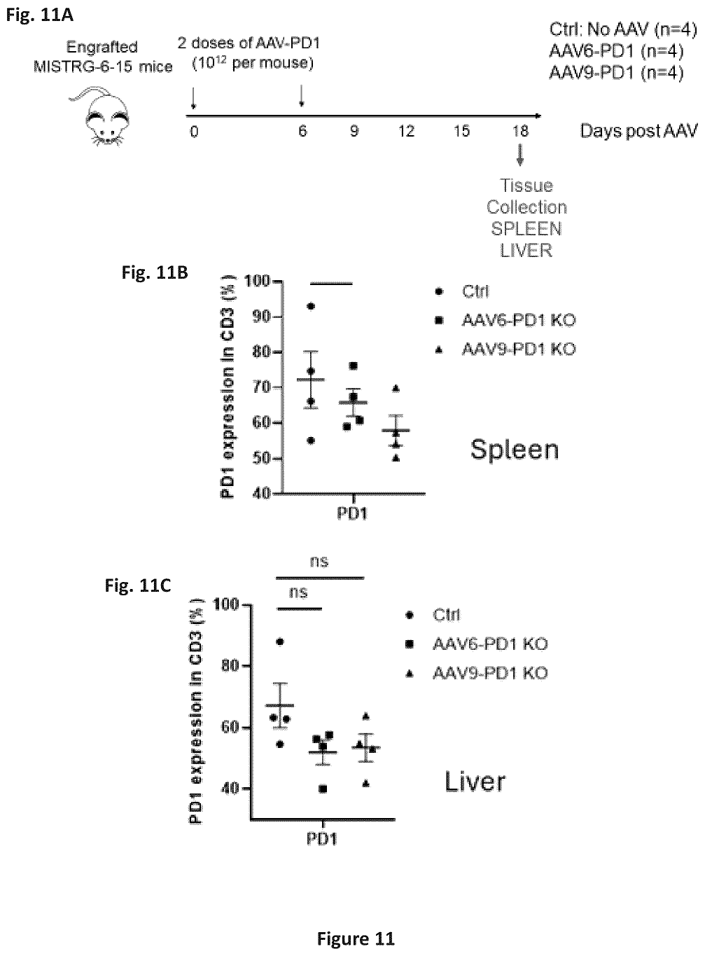

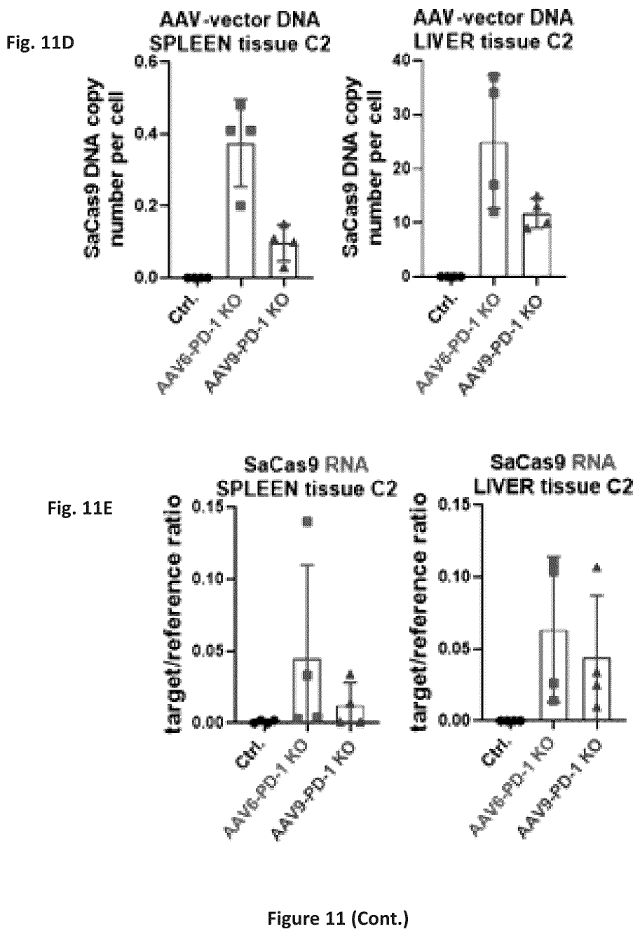

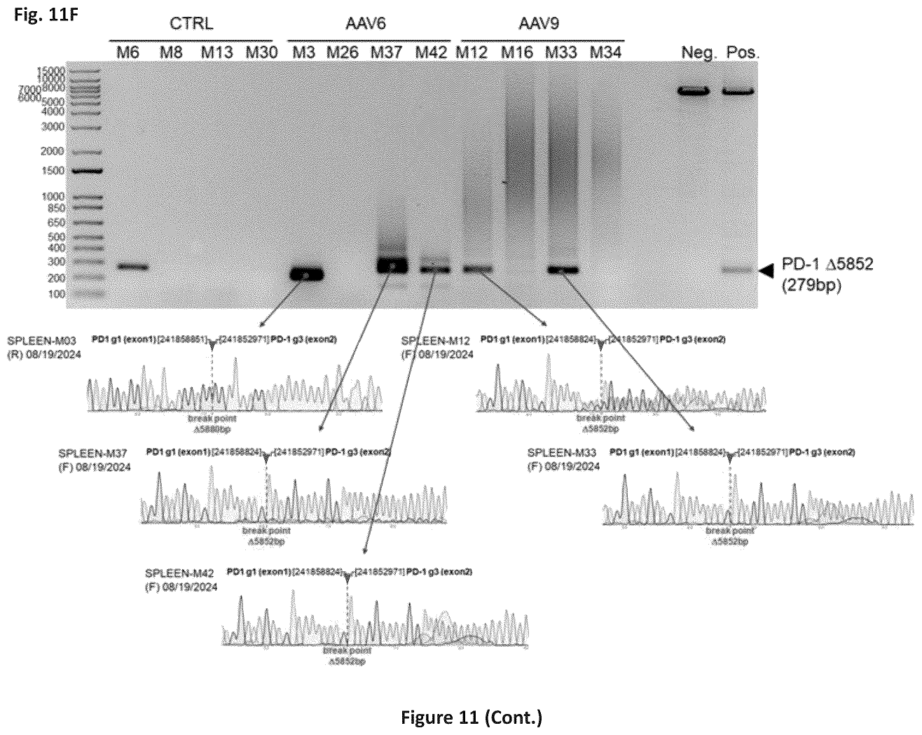

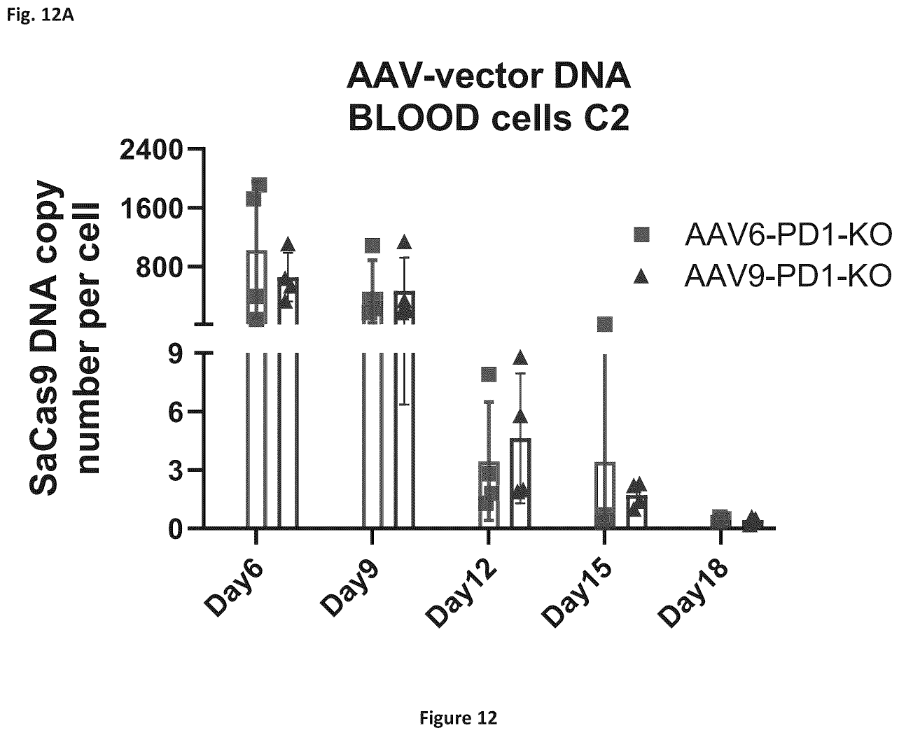

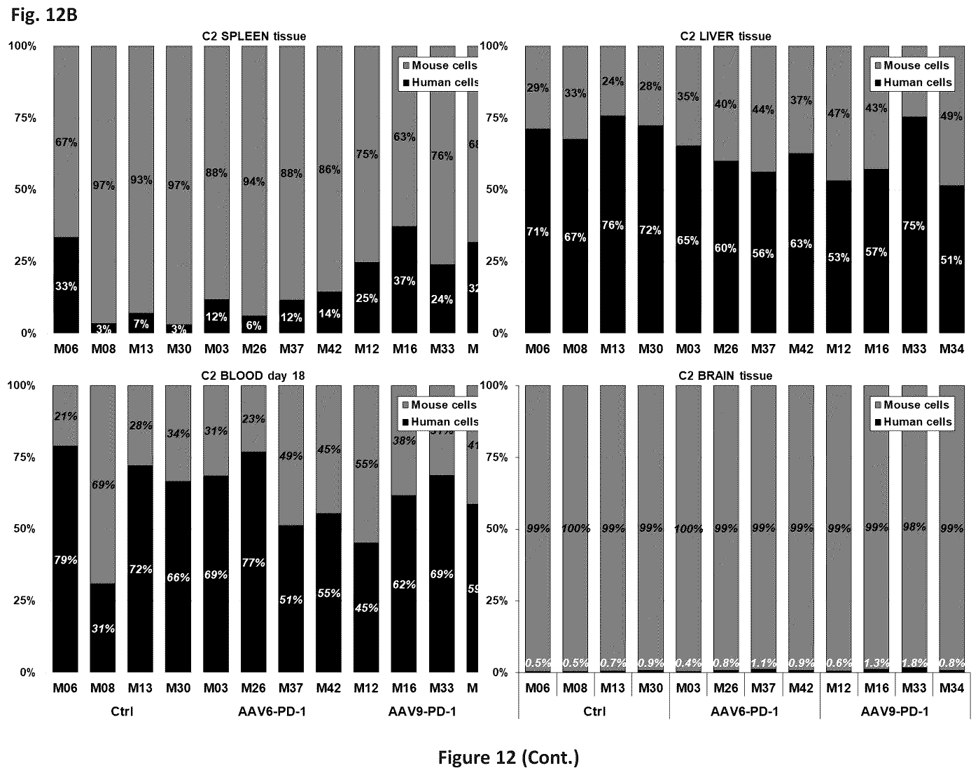

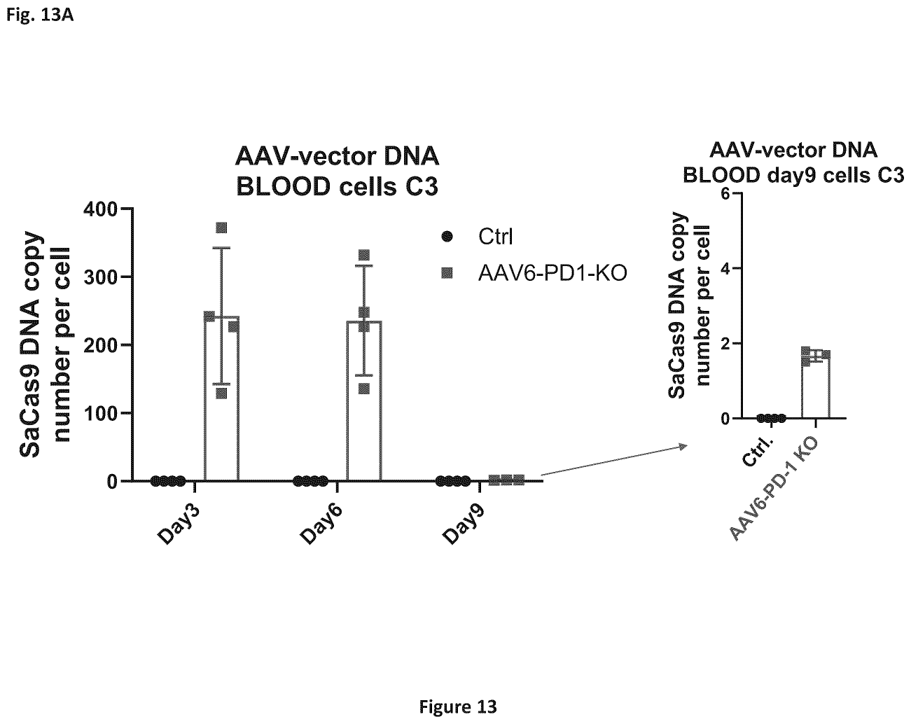

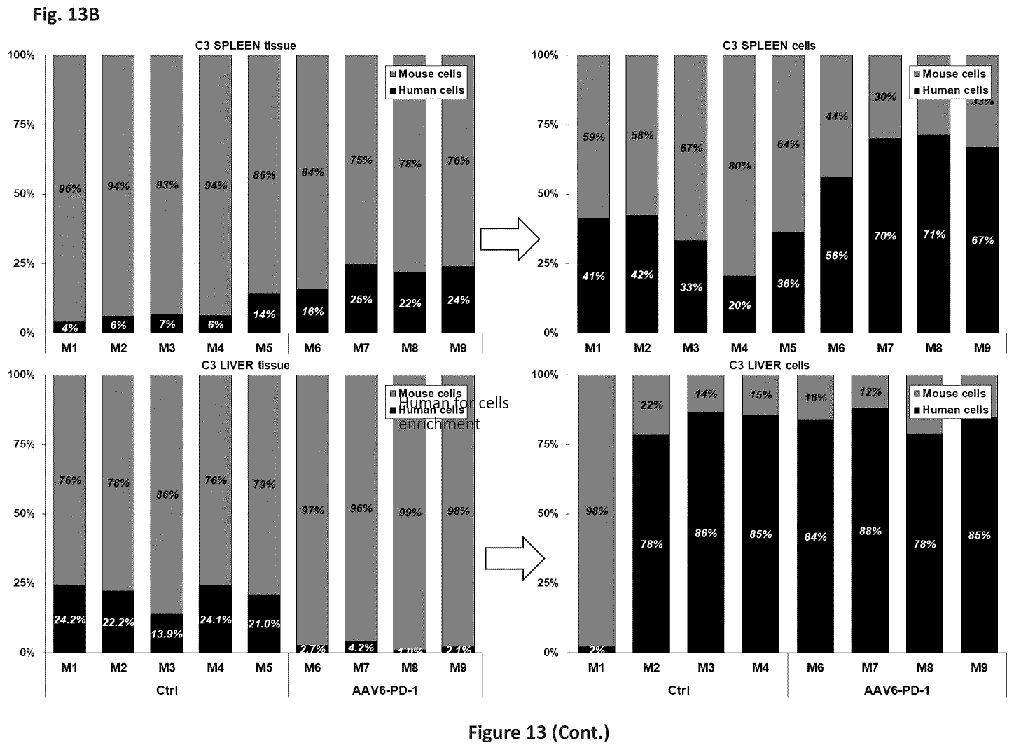

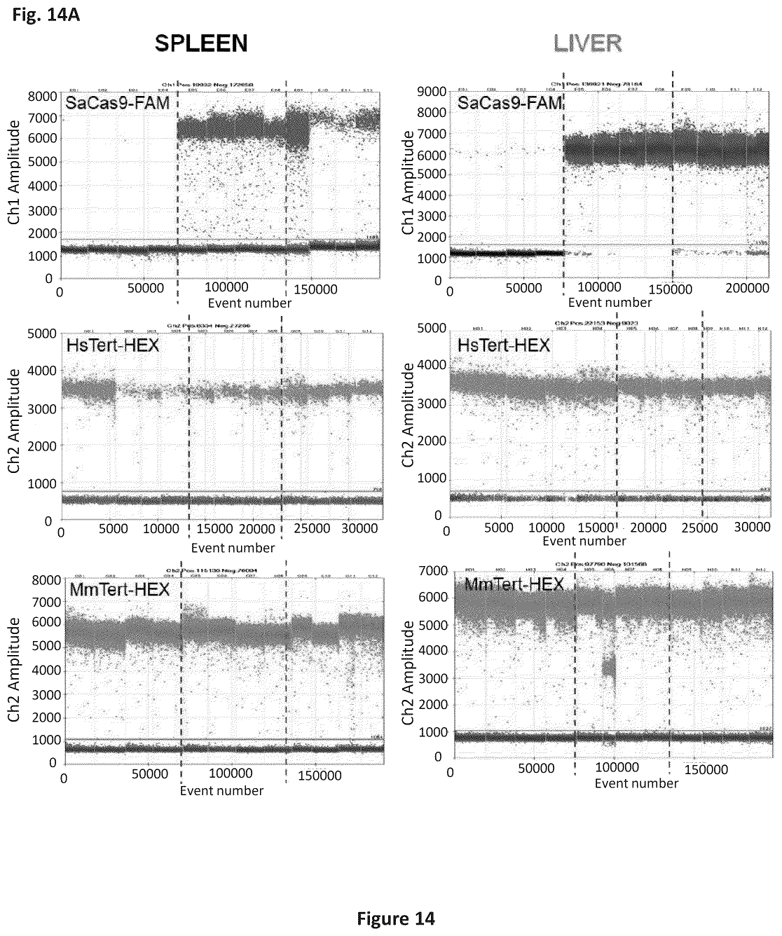

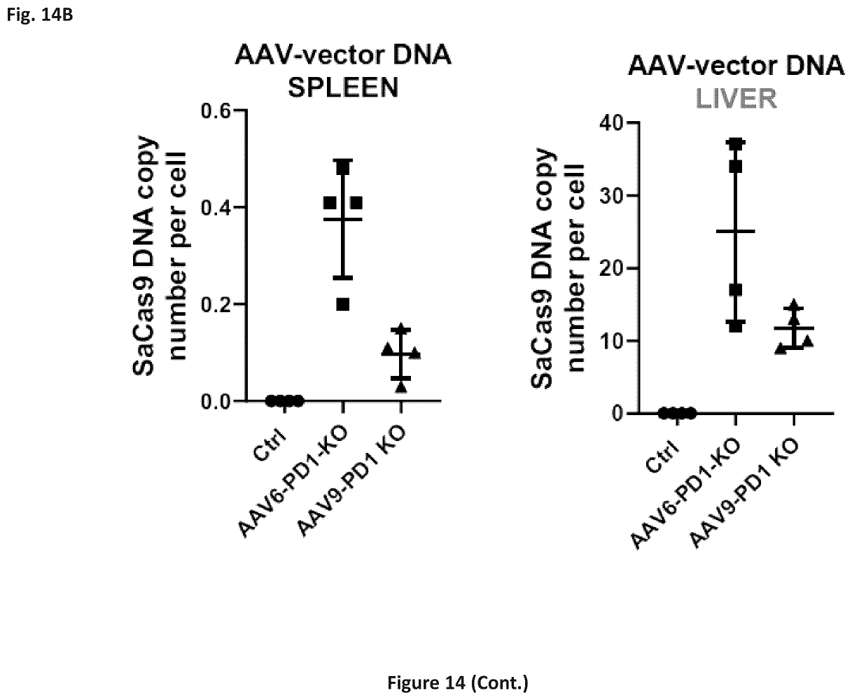

MISTRG-6-15 mice engrafted with human CD34 hematopoietic stem cells were administered two doses of control, AAV6-PD1, or AAV9-PD1 (10¹² VG/mouse total) at day 0 and day 6. After 18 days, the spleen and liver were harvested (FIG. 11A). PD-1 expression was examined in the spleen and liver tissue, with noticeable decreases in PD1 expression in CD3⁺ cells in both the spleen and liver (FIG. 11B). SaCas9 DNA and RNA levels were also examined in the liver, spleen, and blood by ddPCR (FIG. 11C, FIG. 12A, FIG. 13A, and FIG. 14) and RT-ddPCR (FIG. 11D) to confirm successful delivery (FIG. 11C and FIG. 11D). Excision of PD-1 was confirmed by rtPCR and Sanger sequencing (FIG. 11E). Cells were also examined for the proportion of human and mouse cells in each tissue (FIG. 12B and FIG. 12C).

Current immunotherapies targeting PD-1 are based on the use of antibodies to block PD-1 and its ligands PD-L1 and PDL2. It is an arduous process to identify suitable antibodies for therapeutic applications. Antibodies are large, fragile proteins requiring a cold chain and careful handling to maintain their activity. They are difficult to manufacture with good quality control and suffer from batch-to-batch variability. Antibodies have high manufacturing costs. Antibodies may trigger antibody dependent cytotoxicity (ADCC), complement-mediated cytotoxicity (CDC), and other adverse effects. Using CRISPR-Cas9 delivered with AAV bypasses these challenges.

Importantly, the approach of using AAV-compatible SaCas9 (not the 1 kb longer SpCas9) allows the creation of an “all-in-one” AAVCRISPR-PD-1 vector feasible for clinical applications. Adeno-associated viruses (AAVs) are the safest and most effective gene delivery vehicles for gene therapy. Their excellent safety profile and the high efficiency of transduction of a broad range of target tissues, have established AAV vectors as the platform of choice for in vivo gene therapy.

Two gRNAs targeting the coding sequence of the PDCD1 gene increase the chances of inflicting lasting damage to the PDCD1 gene resulting in disruption of PD-1 expression.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A genome editing system comprising:
a) a Cas peptide or a nucleic acid encoding said Cas peptide; and
b) at least one guide RNA (gRNA) molecule comprising a targeting domain that is complementary to a target sequence of a PD-1 gene or at least one nucleic acid encoding said at least one gRNA molecule.

2. The genome editing system of claim 1, wherein the Cas peptide is a Cas9 peptide.

3. The genome editing system of claim 2, wherein the Cas9 peptide is a Staphylococcus aureus Cas9 (SaCas9) peptide.

4. The genome editing system of claim 3, wherein the SaCas9 peptide is an adeno-associated virus (AAV)-compatible SaCas9.

5. The genome editing system of claim 1, wherein said at least one gRNA molecule comprises a nucleotide sequence having at least 95% identity to at least one nucleotide sequence selected from the group consisting of SEQ ID NOs:1-5.

6. The genome editing system of claim 5, wherein said at least one gRNA molecule comprises at least one nucleotide sequence selected from the group consisting of SEQ ID NOs:1-5.

7. The genome editing system of claim 6, wherein said at least one gRNA molecule comprises at least two gRNA molecules,
wherein said at least two gRNA molecules comprise at least two sequences selected from the group consisting of SEQ ID NOs:1-5.

8. The genome editing system of claim 7, wherein said at least two gRNA molecules comprise at least a first gRNA molecule comprising SEQ ID NO:1 and a second gRNA molecule comprising SEQ ID NO:3.

9. A plasmid encoding the genome editing system of claim 1, comprising a nucleotide sequence encoding said Cas peptide and at least one nucleotide sequence encoding said at least one gRNA molecule.

10. The plasmid of claim 9, wherein said Cas peptide is an AAV-compatible SaCas9 peptide.

11. The plasmid of claim 9, wherein said at least one gRNA molecule comprises a nucleotide sequence having at least 95% identity to at least one nucleotide sequence selected from the group consisting of SEQ ID NOs:1-5.

12. The plasmid of claim 11, wherein said at least one gRNA molecule comprises a nucleotide sequence selected from the group consisting of SEQ ID NOs:1-5.

13. The plasmid of claim 12, wherein the plasmid encodes at least two gRNA molecules,
wherein said at least two gRNA molecules comprise at least two sequences selected from the group consisting of SEQ ID NOs:1-5.

14. The plasmid of claim 13, wherein said at least two gRNA molecules comprise at least a first gRNA molecule comprising SEQ ID NO:1 and a second gRNA molecule comprising SEQ ID NO:3.

15. An adeno-associated virus (AAV) comprising the plasmid of claim 9.

16. The AAV of claim 15, wherein the AAV is an AAV6 or an AAV9 serotype.

17. A method of decreasing the expression of a PD-1 gene in a cell, comprising contacting the cell with the genome editing system of claim 1.

18. A method of reducing cytotoxic T lymphocyte (CTL) exhaustion in a subject comprising administering to the subject the AAV of claim 16.

19. A method of treating or preventing a disease or disorder in a subject, comprising administering to the subject the AAV of claim 16.

20. The method of claim 19, wherein the subject is infected with a pathogen selected from the group consisting of: human immunodeficiency virus 1 (HIV-1), hepatitis B virus (HBV), hepatitis C virus (HCV), human T-lymphotropic virus 1 (HTLV-1), influenza, Mycobacterium tuberculosis, a Plasmodium species, a Listeria species, a Toxoplasma species, and a Leishmania species.


SEQUENCE LISTING (30 sequences)

SEQ ID NO: 1 - RNA, 21 nt, synthetic construct
  source 1..21: mol_type = other RNA
gacgactggccagggcgcctg

SEQ ID NO: 2 - RNA, 21 nt, synthetic construct
  source 1..21: mol_type = other RNA
tgctacaactgggctggcggc

SEQ ID NO: 3 - RNA, 21 nt, synthetic construct
  source 1..21: mol_type = other RNA
gggggttccagggcctgtctg

SEQ ID NO: 4 - RNA, 21 nt, synthetic construct
  source 1..21: mol_type = other RNA
gccgcccacgacaccaaccac

SEQ ID NO: 5 - RNA, 21 nt, synthetic construct
  source 1..21: mol_type = other RNA
gggcagcctggtgctgctagt

SEQ ID NO: 6 - DNA, 27 bp, synthetic construct
  source 1..27: mol_type = other DNA
gagtactggccaggtagcctgtagaat

SEQ ID NO: 7 - DNA, 27 bp, synthetic construct
  source 1..27: mol_type = other DNA
gagggctagccagggcacctgtcgagt

SEQ ID NO: 8 - DNA, 27 bp, synthetic construct
  source 1..27: mol_type = other DNA
gaaatgtggccagggcgcctgagggac

SEQ ID NO: 9 - DNA, 27 bp, synthetic construct
  source 1..27: mol_type = other DNA
gctgcctggccagggcgactggcggat

SEQ ID NO: 10 - DNA, 27 bp, synthetic construct
  source 1..27: mol_type = other DNA
gacgactggcgagggccccaagggagt

SEQ ID NO: 11 - DNA, 27 bp, synthetic construct
  source 1..27: mol_type = other DNA
ggggcctccagggcccatctggaggat

SEQ ID NO: 12 - DNA, 27 bp, synthetic construct
  source 1..27: mol_type = other DNA
agtgattcctgggcctgtctgctggag

SEQ ID NO: 13 - DNA, 27 bp, synthetic construct
  source 1..27: mol_type = other DNA
ggggtttccagaggctgtcaggggagt

SEQ ID NO: 14 - DNA, 27 bp, synthetic construct
  source 1..27: mol_type = other DNA
gggggatcctgtgcctgactgtggggt

SEQ ID NO: 15 - DNA, 27 bp, synthetic construct
  source 1..27: mol_type = other DNA
gtggtttccaggggctgtgtggggggt

SEQ ID NO: 16 - DNA, 27 bp, synthetic construct
  source 1..27: mol_type = other DNA
ggggaccccagggactgtctgcagagt

SEQ ID NO: 17 - protein, 27 aa, synthetic construct
GGGGGTTTCAGGGCATGTTTCCTGAGT

SEQ ID NO: 18 - DNA, 27 bp, synthetic construct
  source 1..27: mol_type = other DNA
ctgggatccagggtctgtctgagggga

SEQ ID NO: 19 - DNA, 27 bp, synthetic construct
  source 1..27: mol_type = other DNA
ggggtttccagaggctgtcaggggagt

SEQ ID NO: 20 - DNA, 27 bp, synthetic construct
  source 1..27: mol_type = other DNA
gggggcgcccggccctgtctgctgagt

SEQ ID NO: 21 - DNA, 27 bp, synthetic construct
  source 1..27: mol_type = other DNA
ggcccttccatggcctgtctgtagaag

SEQ ID NO: 22 - DNA, 27 bp, synthetic construct
  source 1..27: mol_type = other DNA
aggtctcccagggcctgtctgtgggga

SEQ ID NO: 23 - DNA, 27 bp, synthetic construct
  source 1..27: mol_type = other DNA
ccgggtcccaaggcctgtctgcagggt

SEQ ID NO: 24 - DNA, 26 bp, synthetic construct
  source 1..26: mol_type = other DNA
gggggttttaggcctgtccactggat

SEQ ID NO: 25 - DNA, 27 bp, synthetic construct
  source 1..27: mol_type = other DNA
aggggctccagagcctggctgaagggt

SEQ ID NO: 26 - DNA, 27 bp, synthetic construct
  source 1..27: mol_type = other DNA
ggggattccatgacgtgtctggaggat

SEQ ID NO: 27 - DNA, 27 bp, synthetic construct
  source 1..27: mol_type = other DNA
aggggttccaggcccagtcagaggagt

SEQ ID NO: 28 - DNA, 27 bp, synthetic construct
  source 1..27: mol_type = other DNA
gtgggcaccagggcctggctgtcgggt

SEQ ID NO: 29 - DNA, 27 bp, synthetic construct
  source 1..27: mol_type = other DNA
gggggttcctgggtccctctgaagagt

SEQ ID NO: 30 - DNA, 27 bp, synthetic construct
  source 1..27: mol_type = other DNA
ttgggttccagggcctggctgctggga